(19) United States
(12) Patent Application Publication
(10) Pub. No.: US 2025/0243249 A1
(43) Pub. Date: Jul. 31, 2025

(54) COMPOSITIONS AND METHODS FOR THE ISOLATION AND PURIFICATION OF ANTI-FUNGAL PROTEIN FROM EPICHLOE FESTUCAE AND USE THEREOF FOR REDUCING SYMPTOMS OF DOLLAR SPOT DISEASE IN TARGETED PLANT SPECIES

(71) Applicant: RUTGERS, THE STATE UNIVERSITY OF NEW JERSEY, New Brunswick, NJ (US)

(72) Inventors: Faith C. BELANGER, East Brunswick, NJ (US); Bruce B. CLARKE, Iselin, NJ (US); Patrick FARDELLA, Jersey City, NJ (US)

(73) Assignee: RUTGERS, THE STATE UNIVERSITY OF NEW JERSEY, New Brunswick, NJ (US)

(21) Appl. No.: 18/847,950

(22) PCT Filed: Mar. 17, 2023
(86) PCT No.: PCT/US2023/064658

§ 371 Date: Sep. 17, 2024

Related U.S. Application Data
(60) Provisional application No. 63/320,820, filed on Mar. 17, 2022.

(51) Int. Cl. C07K 14/37; A01N 63/50; A01P 3/00; C12N 1/14; C12R 1/82

(52) U.S. Cl. CPC C07K 14/37; A01N 63/50; A01P 3/00; C12N 1/14; C12R 2001/82

(57) ABSTRACT

Compositions and methods are provided for prevention and treatment of plant diseases with an antifungal protein.

### CROSS-REFERENCE TO RELATED APPLICATION

This application claims priority of U.S. Provisional application No. 63/320,820 filed Mar. 17, 2022, the entire contents being incorporated herein by reference as though set forth in full.

### GOVERNMENT SUPPORT STATEMENT

This invention was made with government support under Hatch Accession Number 1024790 awarded by the USDA National Institute of Food and Agriculture. The government has certain rights in the invention.

### FIELD OF THE INVENTION

The present invention relates to the fields of agriculture and compounds for the inhibition of pathogen growth in target plant species of interest. More specifically, the invention provides methods for robust production, isolation and utilization of Efe-AfpA and compositions containing the same for treatment and prevention of Dollar Spot Disease and other diseases.

### BACKGROUND OF THE INVENTION

Several publications and patent documents are cited throughout the specification in order to describe the state of the art to which this invention pertains. Each of these citations is incorporated herein by reference as though set forth in full.

Strong creeping red fescue (Festuca rubra subsp. rubra) is a commercially important low-maintenance turfgrass and is often naturally infected with the fungal endophyte Epichloë festucae [Schardl, C. L. et al. 2013; Tadych, M. et al. (2014)]. Epichloë spp. are endophytes of several cool-season grass species, often conferring insect resistance to the grass hosts due to the production of toxic alkaloids. For turfgrasses, cultivars containing Epichloë endophytes are desired because of the enhanced insect resistance they impart [Funk et al. 1993]. In addition to insect resistance, a unique feature of the strong creeping red fescue/E. festucae symbiosis is the endophyte-mediated disease resistance to the causal agent of dollar spot disease, the fungal pathogen Clarireedia jacksonii (formerly Sclerotinia homoeocarpa) [Clarke, B. B. et al. 2006][Salgado-Salazar, C. et al. 2018]. Such endophyte-mediated disease resistance is not seen in the cultivated grasses ryegrass (Lolium perenne L.) or tall fescue (Lolium arundinaceum) [Tian, Z et al. 2017a]. As discussed previously, there is conflicting literature on Epichloë spp. mediated disease resistance in various grass hosts but no field-level resistance has been reported other than for the fine fescues.

The mechanism underlying the unique endophyte-mediated disease resistance in strong creeping red fescue is not yet established. The purified E. festucae antifungal protein, Efe-AfpA, was shown to inhibit the growth of C. jacksonii and to cause membrane damage in plate assays. The antifungal protein gene, Efe-afpA (gene model EfM3.0636600, on the world wide web at csbio-1.csr.uky.edu/endophyte/; GenBank accession MG925781.1) found in E. festucae infecting strong creeping red fescue is not present in most Epichloë genomes for which whole genome sequence is available, being present only in E. baconii, E. aotearoae, E. coenophiala, and E. inebrians.

Efe-AfpA has been difficult to isolate and purify in abundance. It is an object of the invention to provide an improved robust method for production of Efe-AfpA for use in agricultural formulations for controlling fungal infection, particularly in turf grasses.

### SUMMARY OF THE INVENTION

The present invention provides a composition for treating, preventing or controlling fungal disease, damage or infection in plants, particularly in turf grasses. Specifically, the invention relates to a composition comprising an effective amount of antifungal protein, Efe-AfpA in an agriculturally suitable carrier, and to methods for treating, preventing or controlling fungal disease, damage or infection in plants using this composition.

In one aspect, a method for increasing yield of Efe-AfpA antifungal protein from transgenic P. chrysogenum^{Efe-AfpA} is disclosed. An exemplary method comprises culturing conidia from P. chrysogenum^{Efe-AfpA} in high nutrient media for a suitable time period to produce mycelia without inducing Ef-AfpA expression, harvesting and culturing the mycelia of step a) in a minimal low nutrient media for a suitable time period to induce robust Efe-AfpA expression and secretion into said low nutrient media, centrifuging and filtering said low nutrient media to remove conida and excess debris, dialyzing the filtered media of on an ion exchange column, eluting Efe-AfpA with increasing salt concentrations to form an eluant, and filtering and desalting said eluant via passage through filter, thereby isolating said Efe-AfpA antifungal protein.

Also disclosed is an Efe-AfpA containing composition produced by the method of any of the previous claims in a carrier suitable for application to target plants. The carrier can be solid, mineral or liquid and may be applied directly to soil. The composition may also be sprayed or dripped onto target plant species, particularly turf grasses. The composition may also comprise one or more surfactants, one or more water dispersible concentrates, or wettable powders. In certain aspects, the composition comprises at least one fungicide. In other aspects, at least one nematode controlling agent is present.

Method for treating, and, or inhibiting, microbial infection and/or enhancing resistance to a microbial infection comprising administering a composition described above to a target plant also form an aspect of the invention. While the antifungal protein exemplified herein is obtained from E. festucae, antifungal proteins isolated from E. gansuensis var. inebrians. E. baconii, E. aotearoae, and E. coenophiala and anti-fungal compositions comprising the same are also encompassed by the present invention. In preferred embodiments of the method, the antifungal protein is Efe-AfpA from Epichloe festucae. In particularly preferred embodiments, the fungal disease to be treated is dollar spot disease or red thread disease.

In yet another aspect, a method for increasing yield of Efe-AfpA antifungal protein from transgenic Penicillium ssp. transduced with a nucleic acid encoding Efe-AfpA is disclosed. An exemplary method entails culturing conidia from said transduced Penicillium ssp. in high nutrient media for a suitable time period to produce mycelia without inducing Ef-AfpA expression, harvesting and culturing the mycelia in a minimal low nutrient media for a suitable time period to induce robust Efe-AfpA expression and secretion into said low nutrient media, centrifuging and filtering said low nutrient media to remove conida and excess debris, dialyzing said filtered media of step c) on an ion exchange column, eluting said Efe-AfpA with increasing salt concentrations to form an eluant, and filtering and desalting said eluant via passage through filter, thereby isolating said Efe-AfpA antifungal protein. In another aspect, an endogenous antifungal protein has been deleted or mutated in said Penicillium sp. Compositions comprising the Efe-AfpA so produced are also within the scope of the invention.

### BRIEF DESCRIPTION OF THE DRAWINGS

FIG. 1. Percent Growth of PAF and Efe-AfpA-Treated Penicillium chrysogenum conidia. Percent growth was calculated by measuring the change in absorbance of 2×10⁶ conidia mL⁻¹ of Penicillium chrysogenum after treatment with either water, the P. chrysogenum antifungal protein PAF, or the Epichloë festucae antifungal protein Efe-AfpA at a concentration of 5 μg mL⁻¹. The data presented are the percent growth of the treated samples relative to the growth of the water control. OD₆₂₀ absorbance was measured at 0 hr and 24 hr. Standard deviation represented by error bars.

FIG. 2. Microscopy of PAF and Efe-AfpA Treated Penicillium chrysogenum conidia. A. Penicillium chrysogenum mycelium after water treatment. B. Penicllium chrysogenum mycelium after PAF treatment. C. Penicllium chrysogenum mycelium after Efe-AfpA treatment. All samples were observed under brightfield microscopy.

FIG. 3. SDS polyacrylamide gel of purification of Efe-Afp produced in P. chrysogenum. Lane 1, Bio-Rad Precision Plus Protein Dual Xtra Standards; Lane 2, Crude supernatant of Penicillium chrysogenum expressing Efe-AfpA; Lane 3, purified Efe-AfpA.

FIG. 4. Application of purified E. festucae antifungal protein Efe-Afp can protect creeping bentgrass plants from dollar spot infection.

FIG. 5. Purified Efe-AfpA or PAF was incorporated into agar plates and inoculated with plugs of the red thread fungus.

FIG. 6A-6D. Activity of Efe-AfpA and PAF against B. cinerea conidial growth. FIG. 6A.

Growth inhibition of B. cinerea conidia treated with increasing concentrations of either Efe-AfpA or PAF incubated at room temperature for 30 h. The data presented are the means and standard deviations of three replicates. FIG. 6B-6D. Microscopy of B. cinerea conidia treated with water or 0.6 μg mL⁻¹ of either Efe-AfpA or PAF. Bars are 750 μm.

FIG. 7A-7B. Effect of Efe-AfpA and PAF on Botrytis cinerea growth. B. cinerea spores were plated onto PDA amended with increasing concentrations of the antifungal proteins and photographed after 72 h at room temperature.

FIG. 8A-8D. Activity of Efe-AfpA and PAF against Co. cereale conidial growth. FIG. 8A. Growth inhibition of Co. cereale conidia treated with increasing concentrations of either Efe-AfpA or PAF incubated at room temperature for 48 h. The data presented are the means and standard deviations of three replicates. B. Microscopy of Co. cereale conidia treated with water or 1.2 g mL⁻¹ of either Efe-AfpA or PAF. Bars are 750 μm.

FIG. 9A-9B. Effect of Efe-AfpA and PAF on Colletotrichum cereale growth. Co. cereale spores were plated onto PDA amended with increasing concentrations of the antifungal proteins and photographed after 72 h at room temperature.

FIG. 10A-10B. Activity of Efe-AfpA and PAF against Cr. parasitica mycelial growth. Cr. parasitica mycelial plugs were subcultured onto PDA plates amended with 0.5 g mL⁻¹ of Efe-AfpA or PAF. The colony diameters were measured daily. The data presented are the means and standard deviations of three replicates.

FIG. 11. Effect of Efe-AfpA on Cryphonectria parasitica EP155 growth. Cr. parasitica mycelial plugs were subcultured onto PDA amended with increasing concentrations of Efe-AfpA and incubated at room temperature. The colony diameters were measured daily.

FIG. 12. Effect of PAF on Cryphonectria parasitica EP155 growth. Cr. parasitica mycelial plugs were subcultured onto PDA amended with increasing concentrations of PAF and incubated at room temperature. The colony diameters were measured daily.

FIG. 13A-13D. Activity of Efe-AfpA and PAF against F. graminearum conidial growth. FIG. 13A. Growth inhibition of F. graminearum conidia treated with increasing concentrations of either Efe-AfpA or PAF incubated at room temperature for 30 h. The data presented are the means and standard deviations of three replicates. FIG. 13B-13D. Microscopy of F. graminearum conidia treated with water or 10 μg mL⁻¹ of either Efe-AfpA or PAF. Bars are 750 m.

FIG. 14A-14B. Effect of Efe-AfpA and PAF on Fusarium graminearum growth. F. gramineaerum spores were plated onto PDA amended with increasing concentrations of the antifungal proteins and photographed after 72 h at room temperature.

FIG. 15A-15D. Activity of Efe-AfpA and PAF against Py. oryzae conidial growth. FIG. 15A. Growth inhibition of Py. oryzae conidia treated with increasing concentrations of either Efe-AfpA or PAF incubated at room temperature for 48 h. The data presented are the means and standard deviations of three replicates. FIG. 15B-15D. Microscopy of Py. oryzae conidia treated with water or 100 g mL⁻¹ of either Efe-AfpA or PAF. Bars are 750 m.

FIG. 16A-16B. Effect of Efe-AfpA and PAF on Pyricularia oryzae growth. Py. oryzae spores were plated onto PDA amended with increasing concentrations of the antifungal proteins and photographed after 96 h at room temperature.

FIG. 17A-17B. Activity of Efe-AfpA and PAF against L. fuciformis mycelial growth. L. fuciformis mycelial plugs were subcultured onto PDA plates amended with increasing concentrations of Efe-AfpA or PAF. The colony diameters were measured daily. The data presented are the means and standard deviations of three replicates.

FIG. 18A-18B. Effect of Efe-AfpA(FIG. 18A) and PAF (FIG. 18B) on Laetisaria fuciformis growth. L. fuciformis mycelial plugs were subcultured onto PDA amended with increasing concentrations of Efe-AfpA(FIG. 18A) or PAF (FIG. 18B) and incubated at room temperature. The colony diameters were measured daily.

### DETAILED DESCRIPTION OF THE INVENTION

Control of dollar spot disease on creeping bentgrass is a major problem for golf course managers and currently relies heavily on fungicide applications which are often toxic to the environment. Ongoing efforts to address this problem have focused on breeding tolerant cultivars and on improving management protocols. The present invention relates to non toxic compositions and methods for controlling dollar spot resistance in fungal endophyte-infected (Epichloë festucae) strong creeping red fescue. Endophyte-mediated disease resistance is well established in fine fescues (Bonos et al., 2005; Clarke et al., 2006), but is not a general feature of other endophyte-infected grasses such as perennial ryegrass or tall fescue. In efforts to identify and characterize the mechanism and cellular factors responsible for endophyte-mediated disease resistance in fine fescues, these factors can then be used to advantage in formulations suitable for other turfgrasses such as creeping bentgrass, which are not infected with the protective Epichloë endophytes.

### Definitions

An “endophyte” or “endophytic microbe” is an organism that lives within a plant or is otherwise associated therewith. Endophytes can occupy the intracellular or intercellular spaces of plant tissue, including the leaves, stems, flowers, fruits, seeds, or roots. An endophyte can be either a bacterial or a fungal organism that can confer a beneficial property to a plant such as an increase in yield, biomass, resistance, or fitness in its host plant. As used herein, the term “microbe”, “fungus” or “bacteria” is sometimes used to describe an endophyte.

Particular formulations to be applied in spraying forms such as water dispersible concentrates or wettable powders may contain surfactant such as wetting and dispersing agents, e.g., the condensation product of formaldehyde with naphthalene sulphonate, an alkyl-aryl-sulphonate, a lignin sulphonate, a fatty alkyl sulphate an ethoxylated alkylphenol and an ethoxylated fatty alcohol.

As used herein the terms “spray” or “spraying” include the technique of applying to an exterior surface an ejected liquid material.

As used herein, the terms “coat” or “coating” include application, typically of a liquid or flowable solid, to an exterior surface such as a seed.

As used herein, a “stabilizer” includes a chemical compound that can be added to a formulation to prolong the stability and/or viability of components of the formulation, a critical aspect of product shelf-stability. A stabilizer can be one of a variety of compounds, such as a dessicant.

As used herein, a “preservative” includes any chemical compound and/or physical conditions that prevent the decomposition of organic constituents of seeds treated with formulations. Chemical preservatives could include, for example, synthetic or non-synthetic antioxidants and physical preservatives could include, for example, refrigeration, freeze-drying or drying.

According to an embodiment the at least one dispersing agent can be in the range of about 2% to about 60% on a dry weight by weight basis. Various dispersing agents are commercially available for use in agricultural compositions, such as those marketed by Rhone Poulenc, Witco, Westvaco, International Speciality products, Croda chemicals, Borregaard, BASF, Rhodia, etc. According to an embodiment the dispersing agents which can be used in the agricultural composition can be chosen from a group comprising polyvinylpyrrolidone, polyvinylalcohol, lignosulphonates, phenyl naphthalene sulphonates, ethoxylated alkyl phenols, ethoxylated fatty acids, alkoxylated linear alcohols, polyaromatic sulfonates, sodium alkyl aryl sulfonates, glyceryl esters, maleic anhydride copolymers, phosphate esters, condensation products of aryl sulphonic acids and formaldehyde, condensation products of alkylaryl sulphonic acids and formaldehyde, addition products of ethylene oxide and fatty acid esters, salts of addition products. of ethylene oxide and fatty acid esters, sulfonates of condensed naphthalene, addition products of ethylene oxide and fatty acid esters, salts of addition products of ethylene oxide and fatty acid esters, lignin derivatives, naphthalene formaldehyde condensates, sodium salt of isodecylsulfosuccinic acid half ester, polycarboxylates, sodium alkylbenzenesulfonates, sodium salts of sulfonated naphthalene, ammonium salts of sulfonated naphthalene, salts of polyacrylic acids, salts of phenolsulfonic acids and salts of naphthalene sulfonic acids. However, those skilled in the art will appreciate that it is possible to utilize other dispersing agents known in the art without departing from the scope of the claims of the present invention.

In some embodiments, the present invention contemplates methods of manually or mechanically combining the antifungal Efe-AfpA protein with an endophyte having beneficial agricultural properties with one or more plant elements, such as a seed, a leaf, or a root, in order to confer an improved agronomic trait or improved agronomic trait potential to said plant element or host plant.

As used herein, an Efe-AfpA protein is “heterologously disposed” when mechanically or manually applied, artificially inoculated or disposed onto or into a plant element, seedling, plant or onto or into a plant growth medium or onto or into a treatment formulation so that the protein is present on or in said plant element, seedling, plant, plant growth medium, or treatment formulation in a manner not found in nature prior to the application of the Efe-AfpA protein, e.g., said combination which is not found in nature.

The compositions provided herein are preferably stable. In one embodiment, the formulation is substantially stable at temperatures between about 0° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the formulation is substantially stable at temperatures between about 4° C. and about 37° C. for at least about 5, 10, 15, 20, 25, 30 or greater than 30 days.

In some embodiments, plants (including seeds and other plant elements) treated in accordance with the present invention are monocots. In some embodiments, plants (including seeds or other plant elements) treated in accordance with the present invention are dicots. In some embodiments, plants treated in accordance with the present invention include, but are not limited to: agricultural row, agricultural grass plants or other field crops: wheat, rice, barley, buckwheat, beans (soybean, snap, dry), corn (grain, seed, sweet corn, silage, popcorn, high oil), cotton, canola, peas (dry, succulent), peanuts, safflower, sunflower, alfalfa hay, forage crops (alfalfa, clover, vetch, and trefoil), berries and small fruits (blackberries, blueberries, currants, elderberries, gooseberries, huckleberries, loganberries, raspberries, strawberries, bananas and grapes), bulb crops (garlic, leeks, onions, shallots, and ornamental bulbs), citrus fruits (citrus hybrids, grapefruit, kumquat, lines, oranges, and pummelos), cucurbit vegetables (cucumbers, melons, gourds, pumpkins, and squash), flowers, bedding plants, ornamentals, fruiting vegetables (eggplant, sweet and hot peppers, tomatillos, and tomatoes), herbs, spices, mints, hydroponic crops (cucumbers, tomatoes, lettuce, herbs, and spices), leafy vegetables and cole crops (arugula, celery, chervil, endive, fennel, lettuce (head and leaf), parsley, radicchio, rhubarb, spinach, Swiss chard, broccoli, Brussels sprouts, cabbage, cauliflower, collards, kale, kohlrabi, and mustard greens), asparagus, legume vegetable and field crops (snap and dry beans, lentils, succulent and dry peas, and peanuts), pome fruit (pears, apples, and quince), root crops (beets, sugarbeets, red beets, carrots, celeriac, chicory, horseradish, parsnip, radish rutabaga, salsify, and turnips), deciduous trees (maple and oak), pine, small grains (rye, wheat, sorghum, millet, stone fruits (apricots, cherries, nectarines, peaches, plums, and prunes), tree nuts (almonds, beech nuts, Brazil nuts, butternuts, cashews, chestnuts, filberts, hickory nuts, macadamia nuts, pecans, pistachios, and walnuts), tuber crops (potatoes, sweet potatoes, yams, artichoke, cassava, and ginger), and turfgrass (turf, sports fields, parks, established and new preparation of golf course tees, greens, fairways and roughs, seed production and sod production).

Preferred target species of agricultural plants include species of Malvaceae (cotton family): Cotton (Gossypium spp.), Okra Abelmoschus esculentus, Cacao (Theobroma cacao), Kenaf (Hibiscus cannabinus) and Kola nut (Cola spp.). Target species also include other dicot crops, including but not limited to, Coffee (Coffea spp.), Tobacco (Nicotianatabacum), Potato (Solanum tuberosum), Tomato (Solanum lycopsersicum), Sweet potato (Ipomoea batatas), Beans (Phaseolus spp.), Soybeans (Glycine max), Sunflowers (Helianthus spp.) and Rapeseed (Brassica napus).

As used herein, an agricultural grass plant includes, but is not limited to, maize (Zea mays), common wheat (Triticum aestivum), spelt (Triticum spelta), einkorn wheat (Triticum monococcum), emmer wheat (Triticum dicoccum), durum wheat (Triticum durum), Asian rice (Oryza sativa), African rice (Oryza glabaerreima), wild rice (Zizania aquatica, Zizania latifolia, Zizania palustris, Zizania texana), barley (Hordeum vulgare), Sorghum (Sorghum bicolor), Finger millet (Eleusine coracana), Proso millet (Panicum miliaceum), Pearl millet (Pennisetum glaucum), Foxtail millet (Setaria italic), Oat (Avena sativa), Triticale (Triticosecale), rye (Secale cereal), Russian wild rye (Psathyrostachys juncea), bamboo (Bambuseae), grasses, including Agrostis spp., Poa spp., Festuca spp., Lolium spp., Cynodon spp., Zoysia spp., Koleria spp., Danthonia spp., or sugarcane (e.g., Saccharum arundinaceum, Saccharum barberi, Saccharum bengalense, Saccharum edule, Saccharum munja, Saccharum officinarum, Saccharum procerum, Saccharum ravennae, Saccharum robustum, Saccharum sinense, or Saccharum spontaneum).

As used herein, a “reference agricultural plant” is an agricultural plant of the same species, strain, or cultivar to which a treatment, formulation, composition or antifungal preparation as described herein is not administered/contacted. Exemplary reference agricultural plants are described herein. A reference agricultural plant, therefore, is identical to the treated plant with the exception of the presence of the antifungal protein and can serve as a control for detecting the effects of the antifungal protein that is conferred to the plant.

A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, kelkis, shoot, bud. As used herein, a “plant element” is synonymous to a “portion” of a plant, and refers to any part of the plant, and can include distinct tissues and/or organs, and may be used interchangeably with the term “tissue” throughout.

“Biomass” means the total mass or weight (fresh or dry), at a given time, of a plant tissue, plant tissues, an entire plant, or population of plants. Biomass is usually given as weight per unit area. The term may also refer to all the plants or species in the community (community biomass).

An “increased yield” can refer to any increase in biomass or seed or fruit weight, seed size, seed number per plant, seed number per unit area, bushels per acre, tons per acre, kilo per hectare, or carbohydrate yield. Typically, the particular characteristic is designated when referring to increased yield, e.g., increased grain yield or increased seed size.

As used herein, “genetically modified” or “genetically altered” means the modified expression of a protein resulting from one or more genetic modifications; the modifications including but not limited to: recombinant gene technologies, induced mutations, and breeding stably genetically modified plants to produce progeny comprising the altered gene product. Transgenic plants producing antifungal proteins described herein are also provided. Transgenic plants producing the antifungal proteins described herein inhibits the growth of a target organism. In certain embodiments, the antifungal protein is present in a concentration that inhibits the growth of the target organism by at least 10%, 15%, 20%, 25%, 30%, 35%, 40%, 45%, 50%, 55%, 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99%, or 100%.

The terms “transform”, “transfect”, “transduce”, shall refer to any method or means by which a nucleic acid is introduced into a cell or host organism and may be used interchangeably to convey the same meaning. Such methods include, but are not limited to, transfection, virus mediated deliver, electroporation, microinjection, biolistic gene delivery, gene editing, PEG-fusion and the like. In certain embodiments, the cell or host organism is a plant, fungi or bacteria. The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “expression” as used herein in the context of a gene product refers to the biosynthesis of that gene product, including the transcription and/or translation of the gene product. Expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, expression or function of the target gene product can be in the context of a comparison between plant cells, organelles, organs, tissues, or plant parts within the same plant or between plants, and includes comparisons between developmental or temporal stages within the same plant or between plants. The terms “antifungal nucleotide” and “antifungal protein” encompasses any polynucleotide or polypeptide sequence that is capable of inhibiting the growth of the target organism, for example, at the level of transcription or translation, or which is capable of inhibiting the function of a target gene product. In certain embodiments, the antifungal protein is present in a concentration that inhibits the growth of the target organism by at least 10%, 15%, 20%, 25%, 30%, 35%, 40%, 45%, 50%, 55%, 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99%, or 100%.

In many instances the nucleotide sequences for use in the methods of the present invention, are provided in transcriptional units for transcription in the plant of interest. A transcriptional unit is comprised generally of a promoter and a nucleotide sequence operably linked in the 3′ direction of the promoter, optionally with a terminator.

“Operably linked” refers to the functional linkage between a promoter and a second sequence, wherein the promoter sequence initiates and mediates transcription of the DNA sequence corresponding to the second sequence. The expression cassette will include 5′ and 3′ regulatory sequences operably linked to at least one of the sequences of the invention.

Generally, in the context of an over expression cassette, operably linked means that the nucleotide sequences being linked are contiguous and, where necessary to join two or more protein coding regions, contiguous and in the same reading frame. In the case where an expression cassette contains two or more protein coding regions joined in a contiguous manner in the same reading frame, the encoded polypeptide is herein defined as a “heterologous polypeptide” or a “chimeric polypeptide” or a “fusion polypeptide”. The cassette may additionally contain at least one additional coding sequence to be co-transformed into the organism. Alternatively, the additional coding sequence(s) can be provided on multiple expression cassettes.

The methods of transgenic expression can be used to express the antifungal protein, described herein, in an organism that does not usually express the antifungal protein. In certain embodiments, the antifungal protein is expressed in a plant, a fungi, or a bacteria. The methods of transgenic expression comprise transforming a plant cell, fungi, or bacteria with at least one expression cassette comprising a promoter that drives expression in the plant operably linked to at least one nucleotide sequence. Methods for expressing transgenic genes in plants, fungi, and bacteria are well known in the art.

DNA constructs or vectors of the invention may be introduced into the genome of the desired host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Similar conventional techniques may be used to transfect fungal and bacterial cells.

Transformed plant cells that are derived by any of the above transformation techniques can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al., Ann. Rev. of Plant Phys. 38:467-486 (1987).

One of skill will recognize that after the expression cassette or vector is stably incorporated in transgenic plants and confirmed to be operable, it can be introduced into other plants by sexual crossing. Any of a number of standard breeding techniques can be used, depending upon the species to be crossed.

Plant, fungi, and bacteria transformants containing a desired genetic modification as a result of any of the above described methods resulting in the expression of the antifungal protein of the invention can be selected by various methods known in the art. These methods include, but are not limited to, methods such as SDS-PAGE analysis, immunoblotting using antibodies which bind to the protein of interest, single nucleotide polymorphism (SNP) analysis, or assaying for the products of a reporter or marker gene, and the like.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. GFP is exemplified herein. The nucleic acid may be either RNA or DNA, linear or circular, single or double stranded, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under predetermined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989)):

$${T}_{m} = 81.5 ° C . + 16.6 Log [ Na + ] + 0.41 ( \% G + C ) - 0.63 ( \% formamide ) - 600 / ♯bp in duplex$$

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 g/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 g/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25, 30, 50, 75 or more nucleotides nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complimentary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded linear or circular nucleic acid molecule that can be infected, transfected or transformed into cells and replicate independently or within the host cell genome. A linear or circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

The introduced nucleic acid may or may not be integrated (covalently linked) into nucleic acid of the recipient cell or organism. In plant cells, for example, the introduced nucleic acid may be maintained as an episomal element or independent replicon, such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

A “replicon” is any genetic element, for example, a plasmid, cosmid, bacmid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993): Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include polyethylene glycol mediated protoplast transformation, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. Therefore, the phrase “a recombinant organism” encompasses a recombinant cell, as well as a eukaryotic and a prokaryotic organism.

Promoters useful in some embodiments of the present invention may be tissue-specific or cell-specific. The term “tissue-specific” as it applies to a promoter refers to a promoter that is capable of directing selective expression of a nucleotide sequence of interest to a specific type of tissue in the relative absence of expression of the same nucleotide sequence of interest in a different type of tissue (e.g., flower vs. root vs. leaf).

In some cases, the present invention contemplates the use of microbes that are “compatible” with agricultural chemicals, for example, the antifungal protein of the invention, a fungicide, an anti-bacterial compound, or any other agent widely used in agricultural which has the effect of killing or otherwise interfering with optimal growth of microbes. As used herein, a microbe is “compatible” with an agricultural chemical when the microbe is modified, such as by genetic modification, e.g., contains a transgene that confers resistance to an herbicide, or is adapted to grow in, or otherwise survive, the concentration of the agricultural chemical used in agriculture. For example, a microbe disposed on the surface of a seed is compatible with the fungicide metalaxyl if it is able to survive the concentrations that are applied on the seed surface.

In some embodiments, an agriculturally compatible carrier can be used to formulate an agricultural formulation or other composition that includes a purified bacterial preparation. As used herein an “agriculturally compatible carrier” refers to any material, other than water, which can be added to a seed or a seedling without causing or having an adverse effect on the seed (e.g., reducing seed germination) or the plant that grows from the seed, or the like.

As used herein, a “portion” of a plant refers to any part of the plant, and can include distinct tissues and/or organs, and is used interchangeably with the term “tissue” throughout.

A “population” of plants, as used herein, can refer to a plurality of plants that were subjected to the same inoculation methods described herein, or a plurality of plants that are progeny of a plant or group of plants that were subjected to the inoculation methods. In addition, a population of plants can be a group of plants that are grown from coated seeds. The plants within a population will typically be of the same species, and will also typically share a common genetic derivation.

A “reference environment” refers to the environment, treatment or condition of the plant in which a measurement is made.

Provided herein are novel compositions, methods, and products related to our invention's ability to overcome the limitations of the prior art in order to provide reliable increases in turf grass vigor, crop yield, biomass, germination, vigor, stress resilience, and other properties to agricultural crops.

In some embodiments, the antifungal protein formulations can confer beneficial properties across a range of concentrations.

Also described herein is a preparation comprising the antifungal protein described above. The preparation further comprises an agriculturally acceptable carrier, and the preparation comprises an amount of protein sufficient to reduce dollar spot infection. In one embodiment, the isolated microbes expressing the antifungal protein are cultured, for example, on semi-synthetic or synthetic growth medium. In one embodiment, the secreted protein is provided as a powder, for example, a lyophilized powder. In another embodiment, the protein is applied in suspension at a suitable concentration. The preparation can be an aqueous solution, an oil-in-water emulsion or water-in-oil emulsion containing a minimum concentration of the antifungal protein.

The synthetic preparation can be of a defined pH range. In one embodiment, the pH of the preparation can be between pH 5.5-6.0, pH 5.75-6.25, pH 6.0-6.5, pH 6.25-6.75, pH 6.5-7.0, pH 6.75-7.25, and pH 7.0-7.5. The pH of the medium can be adjusted using any biologically compatible buffering agent.

The synthetic preparation can also comprise a carrier, such as diatomaceous earth, clay, or chitin, which act to complex with chemical agents, such as control agents.

The synthetic preparation can also comprise an adherent. Such agents are useful for combining the antifungal protein of the invention with carriers that can contain other compounds (e.g., control agents that are not biologic), to yield a coating composition. Such compositions help create coatings around the plan to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated by reference in its entirety.

The synthetic preparation can also contain one or more reagents that promote internalization of the microbe into the plant, and can include any one of the following classes of compounds: a surfactant, an abrasive, an osmoticum, and a plant signaling molecule.

The preparation can also contain a surfactant, such as Tween 20 as exemplified herein. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

The synthetic preparation of a defined osmolality can also be used. In one embodiment, the synthetic preparation has an osmolality of less than about 100 mOsm, for example less than about 75 mOsm, less than about 50 mOsm, or less than about 25 mOsm. In another embodiment, the synthetic preparation has an osmolality of at least 250 mOsm, for example at least 300 mOsm, at least 400 mOsm, at least 500 mOsm, at least 600 mOsm, at least 700 mOsm, at least 800 mOsm, 900 mOsm or greater. The osmolality of the preparation can be adjusted by addition of an osmoticum: the osmoticum can be any commonly used osmoticum, and can selected from the group consisting of: mannitol, sorbitol, NaCl, KCl, CaCl₂, MgSO₄, sucrose, or any combination thereof.

The Efe-AfpA can be obtained from transformed microbes grown in culture, for example, using semi-synthetic or synthetic growth medium. In addition, can be cultured on solid media, for example on petri dishes, scraped off and suspended into the preparation. Microbes at different growth phases can be used. For example, microbes at lag phase, early-log phase, mid-log phase, late-log phase, stationary phase, early death phase, or death phase can be used.

In another aspect, described herein is an agricultural field, including a greenhouse, comprising the population of treated plants described above. In one embodiment, the agricultural field comprises at least 100 plants. In another embodiment, the population occupies at least about 100 square feet of space, wherein at least about 10%, 20%, 30%, 40%, 50%, 60%, 70%, 80%, 90% or more than 90% of the population comprises an effective amount of the applied antifungal protein.

It will also be appreciated by one skilled in the art that a plant may be exposed to multiple types of fungicides or antibacterial compounds, either simultaneously or in succession, for example at different stages of plant growth. Where the target plant is likely to be exposed to multiple fungicidal and/or antibacterial agents, an antifungal protein that is compatible with many or all of these agrichemicals can be applied to the plant. Resistance, or compatibility with an antimicrobial agent can be determined by a number of means known in the art.

The present invention contemplates the use antifungal formulations capable of conferring resistance to fungal pathogens to the host plant. Increased resistance to fungal inoculation can be measured, for example, using any of the physiological parameters presented above, by comparing with reference agricultural plants. In some embodiments, the Efe-AfpA treated plant exhibits increased biomass and/or less pronounced disease symptoms as compared to a reference agricultural plant grown under the same conditions (e.g., grown side-by-side, or adjacent to, the antifungal formulation treated plants, infected with the fungal pathogen).

In other embodiments, the improved parameter can be an increase in overall biomass of the plant or a part of the plant, including its fruit or seed.

In some embodiments, the plant element is a leaf, and the synthetic combination is formulated for application as a foliar treatment.

In some embodiments, the plant element is a seed, and the synthetic combination is formulated for application as a seed coating.

In some embodiments, the plant element is a root, and the synthetic combination is formulated for application as a root treatment.

In some embodiments, the agricultural carrier may be soil or plant growth medium. Other agricultural carriers that may be used include fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, leaf, root, plant elements, sugar cane bagasse, hulls or stalks from grain processing, ground plant material or wood from building site refuse, sawdust or small fibers from recycling of paper, fabric, or wood. Other suitable formulations will be known to those skilled in the art.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the endophytes used, and should promote the ability of the microbial population to survive application on the plant elements and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In the liquid form, for example, solutions or suspensions, antifungal formulation can be mixed or suspended in aqueous solutions. Suitable liquid diluents or carriers include aqueous solutions, petroleum distillates, or other liquid carriers.

Solid compositions can be prepared by dispersing the antifungal protein of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

Systemic fungicides used for treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some embodiments, the endophyte is compatible with at least one of the fungicides selected from the group consisting of: 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), ipconazole, iprobenfos, iprodione, iprovalicarb, isoprothiolane, isopyrazam, isotianil, kasugamycin, kasugamycin hydrochloride hydrate, kresoxim-methyl, mancopper, mancozeb, mandipropamid, maneb, mepanipyrim, mepronil, mercuric chloride, mercuric oxide, mercurous chloride, metalaxyl, mefenoxam, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z071, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, 5-fluorocytosine and profungicides thereof, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, picolinamide UK-2A and derivatives thereof, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, XRD-563, and zarilamide, IK-1140. In still another embodiment, an endophyte that is compatible with an antibacterial compound is used for the methods described herein. For example, the endophyte is compatible with at least one of the antibiotics selected from the group consisting of: Amikacin, Gentamicin, Kanamycin, Neomycin, Netilmicin, Tobramycin, Paromomycin, Spectinomycin, Geldanamycin, Herbimycin, Rifaximin, streptomycin, Loracarbef, Ertapenem, Doripenem, Imipenem/Cilastatin, Meropenem, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), Sulfonamidochrysoidine (archaic), Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin (Rifampin in US), Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, and Trimethoprim.

A fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in a seed composition.

Preferred nematode-antagonistic biocontrol agents include ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium virens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pasirueia usage, Brevibacillus laterosporus strain G4, Pseudomonas fluorescens and Rhizobacteria.

In certain embodiments, a composition described herein may be in the form of a liquid, a slurry, a solid, or a powder (wettable powder or dry powder). In another embodiment, a composition may be in the form of a seed coating. Compositions in liquid, slurry, or powder (e.g., wettable powder) form may be suitable for coating plant elements. When used to coat plant elements, the composition may be applied to the plant elements and allowed to dry. In embodiments wherein the composition is a powder (e.g., a wettable powder), a liquid, such as water, may need to be added to the powder before application to a seed.

In still another embodiment, the methods can include introducing into the soil or onto a plant an effective amount of the antifungal protein. Such methods can include introducing into the soil one or more of the compositions described herein. The inoculum(s) or compositions may be introduced into the soil according to methods known to those skilled in the art. Non-limiting examples include in-furrow introduction, spraying, coating seeds, foliar introduction, etc. In a particular embodiment, the introducing step comprises in-furrow introduction of the inoculum or compositions described herein.

In one embodiment, plant elements may be treated with composition(s) described herein in several ways but preferably via spraying or dripping. Spray and drip treatment may be conducted by formulating compositions described herein and spraying or dripping the composition(s) onto a seed(s) via a continuous treating system (which is calibrated to apply treatment at a predefined rate in proportion to the continuous flow of seed), such as a drum-type of treater. Batch systems, in which a predetermined batch size of seed and composition(s) as described herein are delivered into a mixer, may also be employed.

### Lyophilization Procedure

Freeze drying bacteria expressing Efe-AfpA antifungal protein (lyophilization) is a very well established method for the archiving and long-term storage. Initial reports of freeze drying bacteria can be found in the middle of last century. The approaches used vary widely, but they all following the standard process associated with lyophilization, namely the freezing of the sample, application of a high vacuum, warming of the sample while under vacuum which causes water sublimation, driving off excess water through a drying phase, and finally sealing of the sample to prevent water uptake. This general process is used to preserve bacteria, fungi, yeasts, proteins, nucleic acids, and any other molecules which may be degraded due to the presence of water.

In another approach, the microbes are cultured as described herein and the secreted antifungal protein isolated and then lyophilized. In this approach, the culture media could be filtered and the culture filtrate comprising the antifungal protein dried. The culture filtrate may optionally be dialyzed prior to drying.

In an alternative approach, the antifungal protein may be encapsulated in alginate beads enriched with humic acid as described by Young C C et al., Biotechnol Bioeng. 2006 Sep. 5; 95(1):76-83. Also see “Alginate beads as a storage, delivery and containment system for genetically modified PCB degrader and PCB biosensor derivatives of Pseudomonas fluorescens F113 B” by Power et al., Journal of Applied Microbiology 110, 1351-1358, 2011.

The following materials and methods are provided to facilitate the practice of Example I.

Expression and Purification of Efe-AfpA in Escherichia coli

A starter culture of the Shuffle T7 cells containing the appropriate plasmid in 50 mL LB supplemented with 30 g mL⁻¹ kanamycin was grown overnight at 30° C. with shaking. The following day this was subcultured into 1 L LB plus 30 μg mL⁻¹ kanamycin and shaken at room temperature until an OD₆₀₀ of 0.6 to 0.8 was reached. Efe-AfpA expression was then induced by the addition of 4 mL of 100 mM IPTG with overnight shaking at room temperature until an OD₆₀₀ of about 1 was reached. Recombinant Efe-AfpA proteins were purified by using TALON @Metal Affinity Resin (TakaraBio). First, cells were collected by centrifugation followed by lysis using 100 mL 1× Fast Break Lysis Reagent (Promega) supplemented with 248 L of 5 mg mL⁻¹ DNase I (Sigma-Aldrich), and rotated for 20 minutes to allow for complete lysis. The 6×HIS-SUMO tagged Efe-AfpA proteins were isolated by the addition of pre-equilibrated TALON resin and incubated for 20 minutes with rotation. Unbound proteins and lysate were removed by centrifuging the resin and decanting the supernatant. Resin was then washed in TALON equilibration buffer, applied to a column, and bound SUMO-tagged Efe-AfpA was eluted using TALON Elution Buffer. To further purify the protein and remove imidazole present in the elution buffer, samples were spun through a 30 kDa Amicon® Ultra-15 Centrifugal Filter Unit, concentrated, and buffer exchanged on a 3 kDa Amicon® Ultra-15 Centrifugal Filter Unit (Millipore). Buffer exchange was accomplished using 50 mM NaPO₄ and 300 mM NaCl pH 7. To remove the 6×His-SUMO tag from the modified forms of Efe-AfpA, 100 μg of purified 6×His-SUMO-tagged protein was incubated overnight at 4° C. with 1 unit of SUMO protease (Lucigen, Middleton, WI, USA). The digested Efe-AfpA solution was buffer exchanged again on a 3 kDa Amicon filter to reduce its salt concentration allowing for purification using CM52 CM Cellulose. CM52 was pre-equilibrated with 50 mM NaPO₄ pH 7 for 1 hour and added to the digest solution to batch purify for 3 hours at 25 C. At pH 7, Efe-AfpA (pI>8) is positively charged and binds to the CM-cellulose. The digest solution was applied to a column, washed with excess 50 mM NaPO₄, and Efe-AfpA was eluted with NaCl amended buffer ranging from 0.1 to 0.5 M. These fractions were then concentrated and desalted using a 3 kDa Amicon @Ultra-15 Centrifugal Filter and sterile distilled water, and sterilized by filtering the protein through a 0.2 μm filter.

Expression and Purification of Efe-AfpA in Pichia pastoris

Pichia pastoris^{Efe-AfpA} was streaked onto solid YPD (1% yeast extract, 2% peptone, 2% dextrose, 2% agar) plates amended with 100 μg mL⁻¹ Zeocin, and incubated at 30° C. until single colonies appeared. A single colony was grown in a starter culture of 50 mL BMGY (1% yeast extract, 2% peptone, 100 mM potassium phosphate pH 6, 1.34% YNB, 4×10⁻⁵% biotin, 1% glycerol) at 30° C. with shaking at 200 rpm until an OD₆₀₀ of at least 2 was reached. The culture was then subcultured into 1 L of fresh BMGY and grown at 30° C. with shaking at 200 rpm until the OD₆₀₀ was at least 2. The culture was then pelleted by centrifugation and resuspended in 1 L BMMY (1% yeast extract, 2% peptone, 100 mM potassium phosphate pH 6, 1.34% YNB, 4×10⁻⁵% biotin, 0.5% methanol) to induce expression of the Efe-AfpA protein. The culture was induced daily with the addition of 5 mL of methanol for 5 days. The cultures were then pelleted by centrifugation at 10,000 rpm for 10 minutes, and the culture supernatant was dialyzed against 8 L of 10 mM NaPO₄, 25 mM NaCl, 0.15 mM EDTA, pH 6.6 buffer in SnakeSkin™ Dialysis Tubing (Thermo Scientific). Dialyzed culture supernatant was then applied to a CM52 CM-cellulose column pre-equilibrated in 10 mM NaPO₄, 25 mM NaCl, 0.15 mM EDTA, pH 6.6 buffer. The column was washed with excess buffer and eluted with increasing salt concentrations from 0.1 to 0.5 M NaCl. These fractions were then filtered through a 30 kDa Amicon® Ultra-15 Centrifugal Filter, concentrated and desalted using a 3 kDa Amicon® Ultra-15 Centrifugal Filter and sterile distilled water, and sterilized by filtering the protein through a 0.2 μm filter.

Expression and Purification of Efe-AfpA in Penicillium chrysogenum

P. chrysogenum^{Efe-AfpA} conidia were streaked onto solid PcMM (Penicillium chrysogenum Minimal Media, 0.3% NaNO₃, 0.05% MgSO⁴×7H₂O, 0.05% KCl, 0.005% FeSO₄×7H₂O, 2% Sucrose, 2.5% 1 M Potassium Phosphate Buffer pH 5.8, 0.1% Trace Elements Solution A, 2% agar) plates supplemented with 200 μg mL⁻¹ Nourseothricin and 0.6 μg mL⁻¹ Pyrithiamine from freezer stocks and grown for 4 days at room temperature. Spores were then harvested in Spore Buffer (0.9% NaCl, 0.01% Tween 80), washed twice in spore buffer, and counted using a hemocytometer. 2×10⁸ conidia were inoculated into 200 mL of Aspergillus nidulans Complete Media (0.2% Peptone, 0.1% Yeast Extract, 0.1% N—Z Amine, 2% Glucose, 2% Salt Solution A, 0.1% Trace Element Solution B) and incubated at room temperature for 48 hrs with shaking at 200 rpm. Mycelia were harvested on cheesecloth, washed with sterile distilled water, and subcultured into 200 mL PcMM to induce expression of the Efe-AfpA protein for 72 hrs at room temperature with shaking at 200 rpm. Culture supernatant was filtered through cheesecloth to remove mycelia and any excess debris was pelleted by centrifugation at 10,000 rpm for 10 minutes. Culture supernatant was then applied to a CM52 CM-cellulose column pre-equilibrated in 10 mM NaPO₄, 25 mM NaCl, 0.15 mM EDTA, pH 6.6 buffer. The column was washed with excess buffer and eluted with increasing salt concentrations from 0.1 to 0.5 M NaCl. These fractions were then filtered through a 30 kDa Amicon® Ultra-15 Centrifugal Filter, concentrated and desalted using a 3 kDa Amicon @Ultra-15 Centrifugal Filter and sterile distilled water, and sterilized by filtering the protein through a 0.2 μm filter.

### Solutions:

Trace Element Solution A: 0.1% FeSO₄×7H₂O, 0.9% ZnSO₄, 0.04% CuSO₄, 0.01% MnSO₄, 0.01% H₃BO₃, 0.01% Na₂MoO₄
Trace Element Solution B: 1.3% ZnSO₄, 0.07% CuSO₄, 0.1% MnSO₄, 0.006% Na₂B₄O₇, 0.23% FeSO₄×7H₂O
Salt Solution A: 2.6% KCl, 2.6% MgSO₄, 7.6% KH₂PO₄, 0.2% Chloroform (v/v)

The following materials and methods are provided to facilitate the practice of Example II.

### Fungi and Culture Conditions

Pe. chrysogenum paf, an isolate over-expressing paf, and Pe. chrysogenum^{Efe-AfpA} an isolate expressing Efe-afpA, were maintained on PcMM (Penicillium chrysogenum Minimal Media) [25,26] supplemented with 200 μg mL⁻¹ nourseothricin and 0.6 g mL⁻¹ pyrithiamine.

All other fungi were maintained on potato dextrose agar (PDA). To generate conidia for Pyricularia oryze, a fungal plug grown on PDA was subcultured onto ryegrass-amended plates and grown for at least 2 weeks. Ryegrass-amended plates were made by autoclaving ryegrass clippings, approximately 20 g in 500 mL of water twice. The clippings were removed by filtering through cheesecloth. The volume of the filtered solution was brought back up to 500 mL with water, agar was added to a final concentration of 1.5%, and the solution was autoclaved.

Purification of Efe-AfpA from Pe. chrysogenum^{Efe-AfpA} and PAF from Pe. chrysogenum paf

Protein purification was performed as previously described [25]. Briefly, for purification of Efe-AfpA 2×10⁸ conidia of Pe. chrysogenum^{Efe-AfpA} were added to 200 mL Aspergillus nidulans Complete Media and shaken at 200 rpm for 48 h. The mycelium was then harvested by filtering the culture through cheesecloth, which was then resuspended in 200 mL PcMM and incubated for 72 h while shaking at 200 rpm. The culture supernatant was collected by filtering through cheesecloth to remove mycelia, and any excess debris was pelleted by centrifugation at 10,000 rpm for 10 minutes. The cleared culture supernatant was applied to a carboxymethyl cellulose (CMC52) (Bio-phoretics, Sparks, NV) column pre-equilibrated with 10 mM NaPO₄, 25 mM NaCl, 0.15 mM EDTA, pH 6.6. The column was then washed with excess buffer and Efe-AfpA was eluted with increasing salt concentrations from 0.1 to 0.5 M NaCl. Similarly, for purification of PAF, 2×10⁸ conidia of Pe. chrysogenum paf was added to 200 mL PcMM and shaken for 72 h at 200 rpm. The culture supernatant was then processed as described above for Efe-AfpA.

Eluted fractions from both purifications were evaluated for the presence of the protein utilizing their respective molecular weights and extinction coefficients at A280 measured by using a Nanodrop ND-1000 spectrophotometer (Thermo Fisher Scientific, Waltham, MA). Efe-AfpA molecular weight: 6.278 kDa, extinction coefficient: 5220 M⁻¹ cm⁻¹. PAF molecular weight: 6.25 kDa, extinction coefficient: 4845 M⁻¹ cm⁻¹. Protein containing fractions were filtered through a 30 kDa Amicon Ultra-15 Centrifugal Filter (MilliporeSigma, Burlington, MA) to remove high molecular weight proteins. The proteins were then concentrated and desalted on a 3 kDa Amicon Ultra-15 Centrifugal Filter with sterile distilled water. The protein samples were finally sterilized by filtering through a 0.2 m polyethersulfone syringe filter (Corning Inc., Corning, NY).

### Antifungal Activity Assays

For fungi that easily produced spores (B. cinerea, Co. graminicola, F. graminearum, Py. oyzae), antifungal activities of Efe-AfpA and PAF were assayed in 24 and 96-well plates. For the 24-well plate activity assays, spores were harvested in Spore Buffer (0.9% NaCl, 0.01% Tween 20), washed twice in Spore Buffer, washed once in sterile distilled water, and resuspended in sterile distilled water. Spores were counted using a hemocytometer and diluted to 2×10⁵ conidia mL⁻¹. Five μL of spores was plated onto 500 μL of PDA amended with increasing concentrations of Efe-AfpA or PAF (0, 0.75, 1.5, 3, 6, 12, 25, 500, 100 g mL⁻¹) in a 24-well plate. Plates were incubated for 72 to 96 h, depending on the growth rate of the fungus. Assays were done in duplicate and the experiment was completed twice.

For 96-well activity assays, spores were harvested in Spore Buffer, washed twice in Spore Buffer, washed once in 2×low cation media (LCM) (1×LCM is 2 g L⁻¹ glucose, 0.1 g L⁻¹ yeast extract, 0.05 g L⁻¹ peptone) and resuspended in fresh 2×LCM. Spores were counted on a hemocytometer and diluted to 2×10⁴ conidia mL⁻¹ with 2×LCM. One hundred L of spores and 100 μL of water containing increasing concentrations of Efe-AfpA or PAF were incubated in each well to final concentrations of 0, 0.3, 0.6, 1.2, 5, 10, 20, 30, 40, 50, 100 g mL⁻¹ antifungal protein. Plates were incubated at room temperature for 24 to 48 h, depending on how fast the fungi grew. Growth was monitored at A620 using a microtiter plate reader (Absorbance 96, Byonoy GmbH, Hamburg, Germany). Optical density at A620 was measured at 0 h and subtracted from subsequent readings to correct for background absorbance. The corrected absorbance of untreated control conidia was considered 100% growth. The percent inhibition by each protein was calculated by comparing the conidia growth of the treated samples to the untreated samples. Wells were visualized microscopically (EVOS M5000, Invitrogen). Each treatment had 3 replicates and the experiment was completed twice.

Fungi that did not produce spores easily (Cr. parasitica Ep155, L. fuciformis, R. solani) had their sensitivity to Efe-AfpA and PAF determined using fungal plugs. These fungi were grown from 4 to 10 days on PDA, depending on the growth rate of the fungus, prior to being subcultured. A 5 mm plug of each fungus was placed in the center of 8 mL PDA plates amended with increasing concentrations of Efe-AfpA or PAF (0, 0.5, 1, 10, 20, 30, 40, 50, 100 μg mL⁻¹). Cross sectional fungal diameter was measured, and plates were photographed daily. Each treatment had three replicates and the experiment was completed twice.

The following examples are provided to illustrate certain embodiments of the invention. They are not intended to limit the invention in any way.

### Example I

E. festucae Antifungal Protein (Efe-AfpA) Purification

In order to identify the mechanism of the E. festucae antifungal protein induced resistance to dollar spot in strong creeping red fescue the previously identified antifungal protein was purified and inhibitory activity against the dollar spot fungus determined. We have optimized purification methods for producing large amounts of the protein for direct application to plants.

E. festucae antifungal protein, designated Efe-AfpA, is a factor in the well-documented disease resistance seen in endophyte-infected fine fescues in the field (Bonos et al., 2005; Clarke et al., 2006). We wished to determine if the endophyte antifungal protein can protect creeping bentgrass plants from dollar spot disease, thereby providing an alternative or supplement to synthetic fungicides. To do this requires a substantial amount, probably milligram levels, of purified antifungal protein.

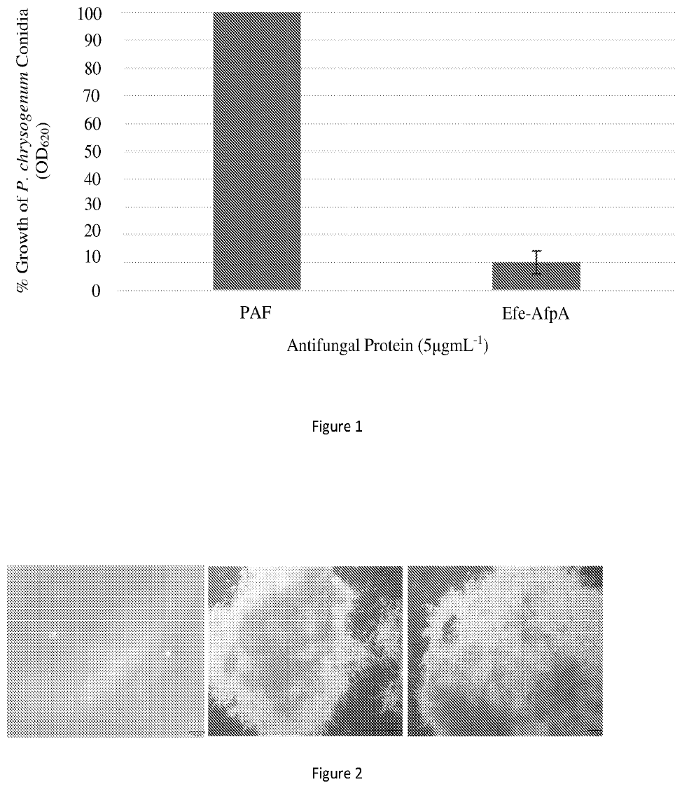

Efe-AfpA was expressed in yeast, in bacteria, and in the fungus Penicillium chrysogenum (Tian et al., 2017; Fardella et al., 2020, 2021, and 2022). Active antifungal protein was purified from all three systems, with the best system being P. chrysogenum. The engineered strain of P. chrysogenum used in these assays has a similar antifungal protein, designated PAF, which was deleted (Marx, 2004; Sonderegger et al., 2016). An unexpected result from expression of Efe-AfpA in P. chrysogenum was the discovery that Efe-AfpA had activity against P. chrysogenum when expressed therein. Although highly active antifungal protein could be recovered from P. chrysogenum, the yields were variable and in some cases lysis of the fungal cells was observed. This result was unexpected because the P. chrysogenum antifungal protein PAF is very similar in sequence to the E. festucae antifungal protein. Moreover, under the same growth conditions, PAF does not have activity against P. chrysogenum. To confirm the impression that Efe-AfpA had activity against P. chrysogenum, the activity was quantitatively determined by measuring growth of conidia of P. chrysogenum. Even at the low concentration of 5 μg mL⁻¹ of Efe-AfpA, growth of P. chrysogenum conidia was dramatically inhibited relative to growth of the PAF-treated sample (FIG. 1). Microscopy of the treated samples is shown in FIG. 2. The P. chrysogenum conidia germinated and grew in the water treated and PAF treated samples, but there was almost no growth in the Efe-AfpA treated sample.

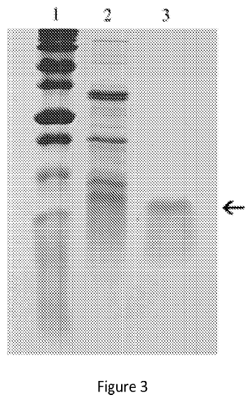

These variable results required the development of a new protocol for growth of P. chrysogenum expressing Efe-AfpA which avoided the toxic effects of the antifungal protein on P. chrysogenum. The improved protocol paired growth of the fungus for 48 hours in a high nutrient medium, conditions which do not induce expression of the antifungal protein, followed by transfer of the fungal mycelium to a low nutrient medium for 48 to 72 hours. In the high nutrient medium, the fungus grew rapidly generating a large biomass of mycelium. In the low nutrient medium, the antifungal protein was expressed and secreted to the surrounding medium from the large biomass, resulting in high yields of active protein. Efe-AfpA was purified from the culture medium using a combination of cation exchange and size exclusion filtration (FIG. 3).

### Application of Purified Antifungal Protein to Dollar-Spot Inoculated Creeping Bentgrass Plants

The overall goal of this project is to determine if application of the purified antifungal protein to dollar spot inoculated creeping bentgrass plants could protect the plants from disease symptoms. To do that we needed to first establish a method of inoculating plants in the greenhouse with the dollar spot fungus. For field assays, the dollar spot fungus is typically grown on sterilized seeds and those seeds are used as inoculum. However, this method can be difficult to standardize for the greenhouse. Therefore, we used agar plugs of the dollar spot fungus to inoculate plants in the greenhouse, since it is easier to precisely control the inoculum. This method was successful in resulting in disease symptoms on the inoculated plants.

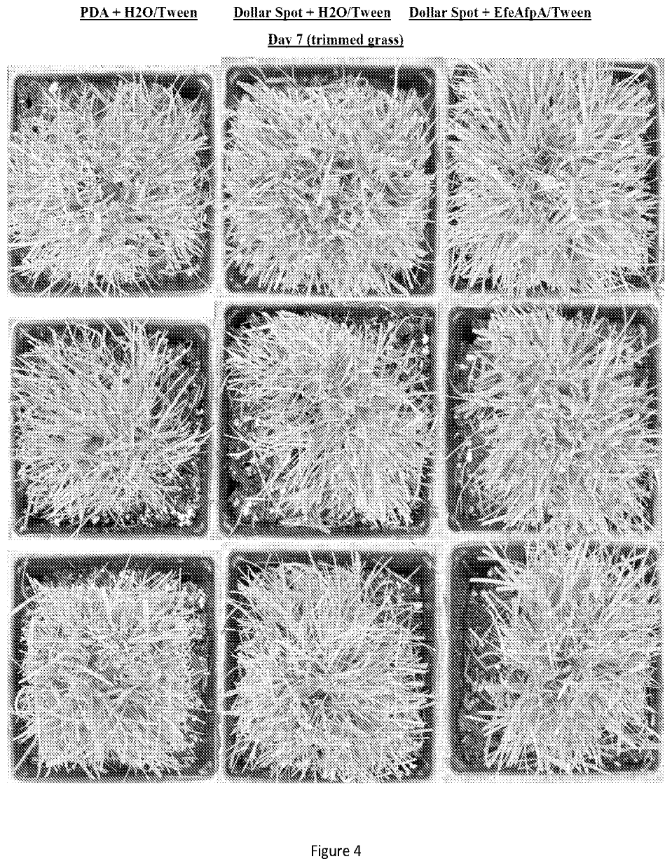

Greenhouse assays were performed to assess the ability of the antifungal protein to protect creeping bentgrass plants from dollar spot disease symptoms. The results are promising and are shown in FIG. 4. In this experiment creeping bentgrass plants were inoculated with agar plugs of the dollar spot fungus or, for the control plants, plugs of plain agar. The experiment was done in triplicate. One set of dollar spot inoculated plants was also sprayed with the antifungal protein in water containing the surfactant tween. The controls and the other set of dollar spot inoculated plants were sprayed with water containing tween. The plants were sprayed daily for 7 days.

As can be seen in FIG. 4 above, the dollar spot inoculated plants exhibited extensive disease symptoms (center lane), whereas the plants sprayed with purified antifungal protein exhibited only minimal disease symptoms (right lane). The control uninoculated plants are in the left lane.

### Activity of the Antifungal Protein Against the Red Thread Pathogen

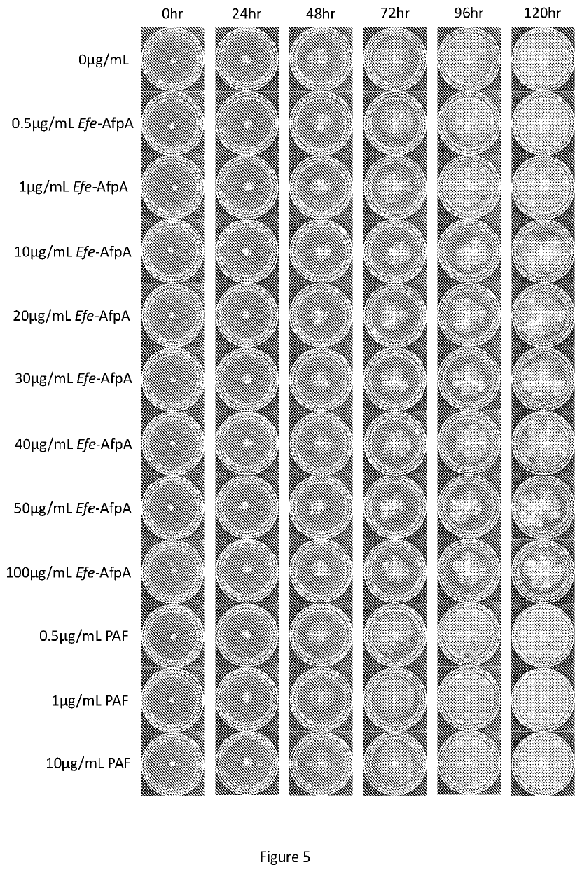

Red thread is a disease caused by the fungal pathogen Laetisaria fuciformis and can be serious problem on fine fescues (Bonos et al., 2005). We have tested the purified antifungal protein against the red thread fungus in a petri dish assay (FIG. 5). Efe-AfpA did have activity against the red thread fungus whereas the similar protein, PAF, from Penicillium chrysogenum did not.

### REFERENCES

Schardl C. L., Young C. A., Hesse U., Amyotte S. G., Andreeva K., Calie P. J., Fleetwood D. J., Haws D. C., Moore N., Oeser B., et al. Plant-symbiotic fungi as chemical engineers: Multi-genome analysis of the Clavicipitaceae reveals dynamics of alkaloid loci. PLoS Genet. 2013; 9: e1003323. doi: 10.1371/journal.pgen.1003323.
Tadych M., Bergen M. S., White J. F., Jr. Epichloë spp. associated with grasses: New insights on life cycles, dissemination and evolution. Mycologia. 2014; 106:181-201.
Funk C. R., White R. H., Breen J. P. Importance of Acremonium endophytes in turfgrass breeding and management. Agric. Ecosyst. Environ. 1993; 44:215-232.
Clarke B. B., White J. F., Jr., Hurley Torres M. S., Sun S. Endophyte-mediated suppression of dollar spot disease in fine fescues. Plant Dis. 2006; 90:994-998.
Salgado-Salazar C., Beirn L. A., Ismaiel A., Boehm M. J., Carbone I., Putman A. I., Tredway L. P., Clarke B. B., Crouch J. A. Clarireedia: A new fungal genus comprising four pathogenic species responsible for dollar spot disease of turfgrass. Fungal Biol. 2018; 122:761-773. doi: 10.1016/j.funbio.2018.04.004. [PubMed][CrossRef][Google Scholar]
Tian, Z., Wang, R., Ambrose, K. V., Clarke, B. B., and Belanger, F. C. (2017a) Isolation of a potential antifungal protein produced by Epichloë festucae, a fungal endophyte of strong creeping red fescue. International Turfgrass Society Research Journal 13: 233-235
Ambrose, K. V., Belanger, F. C. (2012) SOLiD-SAGE of endophyte-infected red fescue reveals numerous effects on host transcriptome and an abundance of highly expressed fungal secreted proteins. PLoS ONE 7(12): e53214
Bonos, S. A., Wilson, M. M., Meyer, W. A., Funk, C. R. (2005) Suppression of red thread in fine fescues through endophyte-mediated resistance. Applied Turfgrass Science 10:1094.
Clarke, B. B., White, J. F. Jr., Hurley, R. H., Torres, M. S., Sun, S., Huff, D. R. (2006) Endophyte-mediated suppression of dollar spot disease in fine fescues. Plant Disease 90:994-998
Fardella, P., Wang, R., Luo S., Clarke, B. B., Belanger, F. C. (2020) Epichloë festucae antifungal protein purification and gene knock-out. Proceedings of the Twenty-Ninth Annual Rutgers Turfgrass Symposium, Jan. 10, 2020
Fardella, P., Clarke, B. B., Belanger, F. C. (2021) Applications of the fungal endophyte Epichloë festucae antifungl protein Efe-AfpA. Proceedings of the Thirtieth Anniversary Rutgers Turfgrass Symposium, Mar. 18, 2021
Marx, F. (2004) Small, basic antifungal proteins secreted from filamentous ascomycetes: a comparative study regarding expression, structure, function and potential application. Applied Microbiology and Biotechnology 65, 1330142
Sonderegger, C, Galgoczy, L., Garrigues, S., Fizil, A., Borics, A., Manzanares, P., Hededus, N., Huber, A., Marcos, J. F., Batta, G., Marx, F. (2016) A Penicillium chrysogenum-based expression system for the production of small, cysteine-rich antifungal proteins for structural and functional analyses. Microbial Cell Factories 15:192
Tian, Z., Wang, R., Ambrose, K. V., Clarke, B. B., and Belanger, F. C. (2017a) Isolation of a potential antifungal protein produced by Epichloë festucae, a fungal endophyte of strong creeping red fescue. International Turfgrass Society Research Journal 13: 233-235
Tian, Z., Wang, R., Ambrose, K. V., Clarke, B. B., and Belanger, F. C. (2017b) The Epichloë festucae antifungal protein has activity against the plant pathogen Sclerotinia homoeocarpa, the causal agent of dollar spot disease. Scientific Reports 7:5643
Tian, Z., Wang, R., Clarke, B. B., and Belanger, F. C. (2017c) A fungal endophyte vs. the dollar spot pathogen. Cutting Edge in Golf Course Management, 09.17: 79

### Example II

### Antifungal Activity of Efe-AfpA Against Numerous Plant Pathogens

Fungal plant pathogens can present major problems for most crop species. Currently, control of fungal diseases relies heavily on the use of fungicides. However, there are problems associated with fungicide use, including potential toxicity to non-target organisms and development of resistance in the target fungus. An antifungal protein, Efe-AfpA, from the fungal endophyte Epichloë festucae, is shown herein as having inhibitory activity against important plant pathogens. These results indicate that Efe-AfpA is effective as a biofungicide to target a broad range of destructive plant pathogens.

Botrytis cinerea

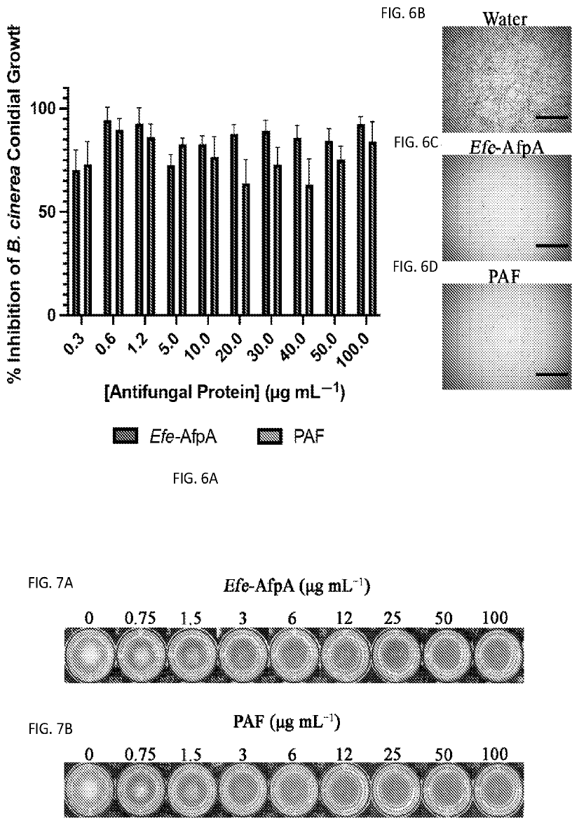

Botrytis cinerea is a necrotrophic ascomycete responsible for gray mold on over 200 crop species worldwide. While it causes disease issues in the field and in green-houses, it is also a considerable post-harvest problem because the fungus can remain quiescent for extended periods of time before becoming active [27]. B. cinerea was listed as the second most important fungal plant pathogen and causes between $10 to $100 billion in losses annually worldwide [28, 29]. Here, Efe-AfgA had a minimal inhibitory concentration (MIC) against B. cinerea of 0.6 μg mL⁻¹ (FIG. 6, Table 1, FIG. 7). MIC is defined as the minimal concentration resulting in at least 90% inhibition of growth of the target organism. PAF did have activity against B. cinerea but its activity did not result in 90% inhibition at any of the concentrations tested (FIG. 6, Table 1).

TABLE 1
Minimal and maximum inhibitory concentrations of
Efe-AfpA and PAF against fungal plant pathogens.

| | Minimal Inhibitory Concentration¹ | | | | Maximum Inhibitory Concentration² | | | |
|---|---|---|---|---|---|---|---|---|
| | Efe-AfpA | | PAF | | Efe-AfpA | | PAF | |
| | μg | % | μg | % | μg | % | μg | % |
| Organism | mL⁻¹ | Inhibition | mL⁻¹ | Inhibition | mL⁻¹ | Inhibition | mL⁻¹ | Inhibition |
| Ascomycetes | | | | | | | | |
| B. cinerea | 0.6 | 94.4 | — | — | 0.6 | 94.4 | 0.6 | 89.6 |
| Co. cereale | 1.2 | 92.9 | 40 | 95.6 | 40 | 96.6 | 100 | 96.4 |
| Cr. parasitica | 0.5 | 98.8 | 0.5 | 100 | 0.5 | 98.8 | 0.5 | 100 |
| F. graminearum | 10 | 91 | — | — | 100 | 98.7 | 100 | 75.7 |
| P. oryzae | — | — | — | — | 100 | 87.1 | 20 | 75.3 |
| Basidiomycetes | | | | | | | | |
| L. fuciformis | — | — | — | — | 100 | 46.5 | — | — |

¹The mimimal inhibitory concentration is defined as the concentration needed for 90% inhibition. “—” indicates activity did not result in 90% inhibition at any of the concentrations tested
²The maximum inhibitory concentration is defined as the highest inhibition obtained at any concentration tested. “—” indicates activity did not result in inhibition at any of the concentrations tested

Colletotrichum cereale

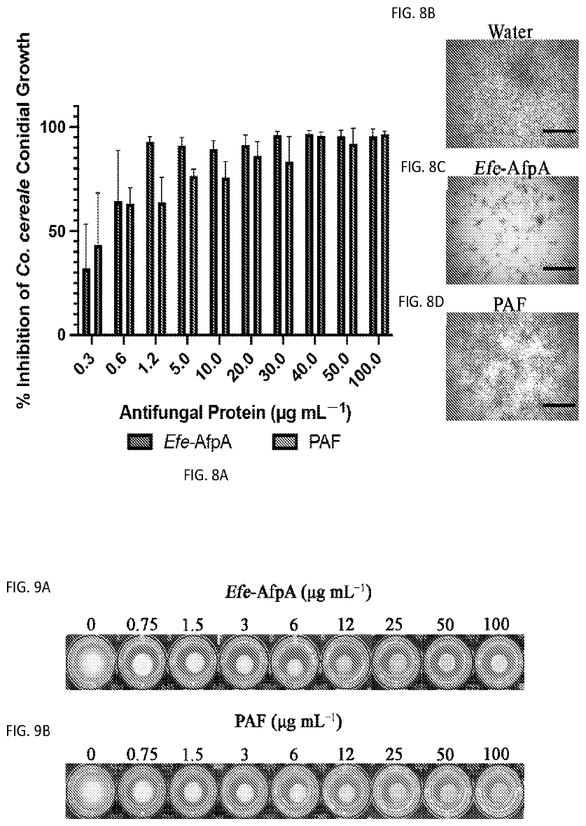

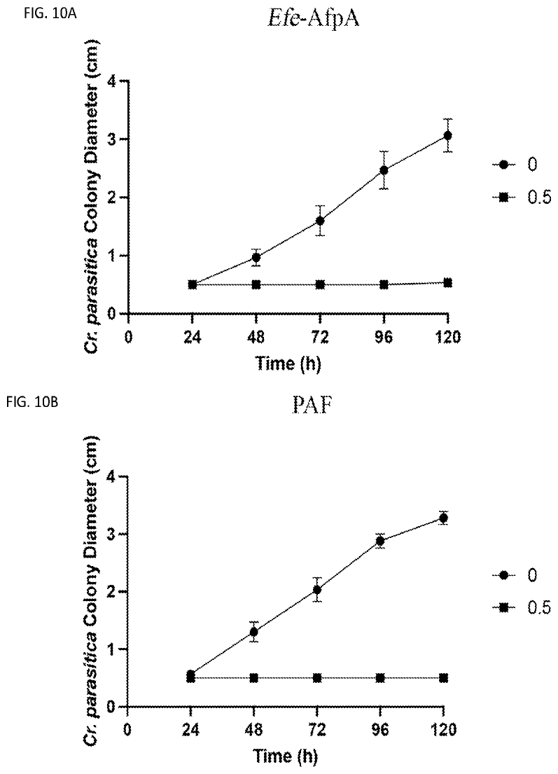

Colletotrichum spp. are common and destructive pathogens on many plant species and are particularly destructive on most agricultural crops. They are ranked the eighth most important fungal pathogens of plants [28]. Co. cereale is responsible for anthracnose on turfgrasses such as Poa annua and Agrostis species, where the disease occurs as either a foliar blight or basal rot [33]. Both Efe-AfpA and PAF had activity against Co. cereale that resulted in greater than 90% inhibition, with MIC concentrations of 1.2 and 40 μg mL⁻¹, respectively (FIG. 8, Table 1, FIG. 9).

Cryphonectria parasitica

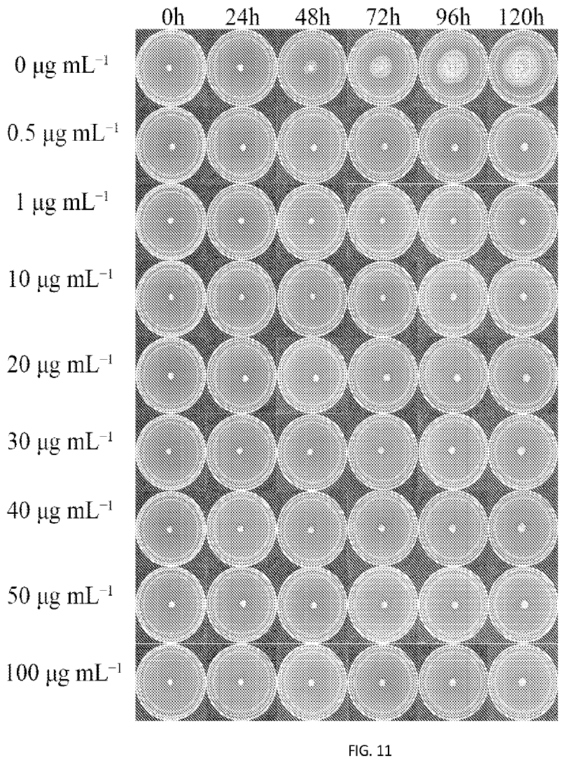

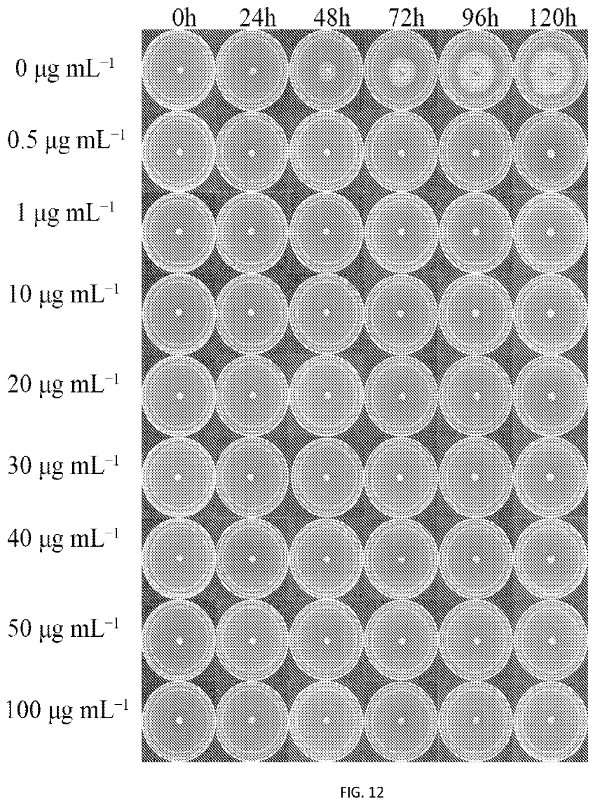

Cryphonectria parasitica is the causal agent of chestnut blight, which nearly eliminated the American chestnut tree (Castanea dentata) worldwide [34]. Current methods of alleviating the disease and restoring the American chestnut to its native range include development of interspecific hybrids, backcross breeding, and genetic engineering [35]. Since Cr. parasitica produces spores slowly, taking 3 to 4 weeks [36], the inhibitory activity of the antifungal proteins was determined in 8 mL agar plate assays using fungal mycelium plugs. Both Efe-AfpA and PAF were active against Cr. parasitica, with the same MIC value of 0.5 g mL⁻¹ (FIG. 10, Table 1, FIGS. 11 and 12). Since there was nearly complete inhibition at the lowest concentration tested, 0.5 g mL-1, higher concentrations are not shown in the graphs in FIG. 10.

Fusarium graminearum

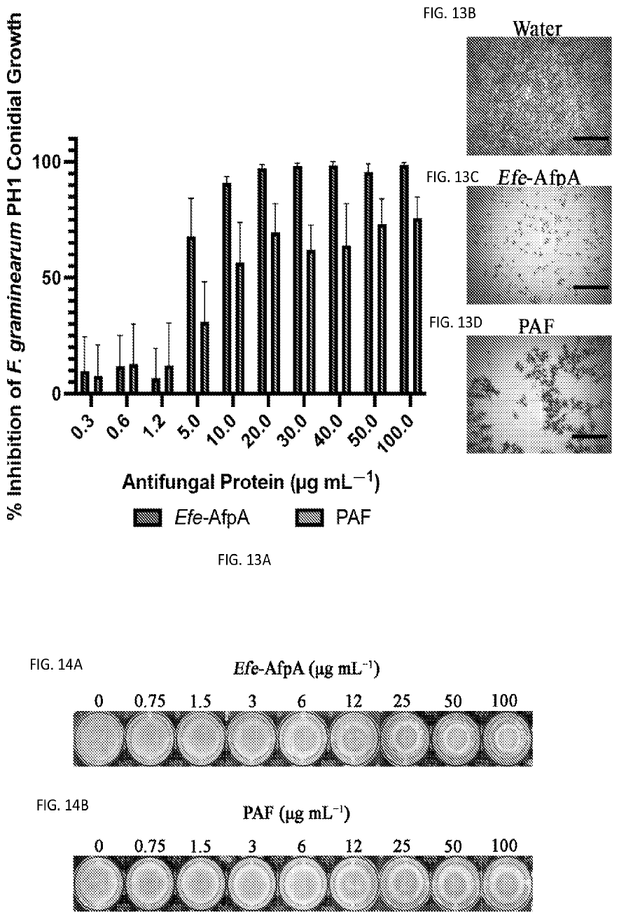

Fusarium graminearum causes both Fusarium head blight on wheat and barley. Additionally, Fusarium stalk and car rot on maize and was considered the fourth most important fungal plant pathogen [28,37]. In addition to its destructive impact on wheat and barley, F. graminearum produces mycotoxins detrimental to human and animal health, such as deoxynivalenol (DON) [38]. Both Efe-AfpA and PAF had activity against F. graminearum, but only Efe-AfpA had activity that resulted in greater than 90% inhibition, with a MIC of 10 μg mL⁻¹ (FIG. 13, Table 1, FIG. 14).

Pyricularia oryzae

Pyricularia oryzae (previously Magnaporthe oryzae) is the causal agent of rice blast and was voted the number one fungal plant pathogen from a scientific and economic perspective [28,39]. Rice (Oryza sativa) is an economically and agriculturally important crop that feeds about half the world's population, and about 10 to 30% yield can be lost annually due to rice blast disease [40]. Py. oryzae also causes gray leaf spot of many genera of turfgrass including Cynodon, Eremochloa, Festuca, Lolium, Paspalum, Pennisetum, and Stenotaphrum [33]. Gray leaf spot was also recently identified on the turfgrass Festuca brevipila (hard fescue), which had not previously been reported to be sensitive to Py. oryzae [41]. As the pathogen presents problems on both rice and turf, prior to these experiments, new mechanisms of plant protection were needed.

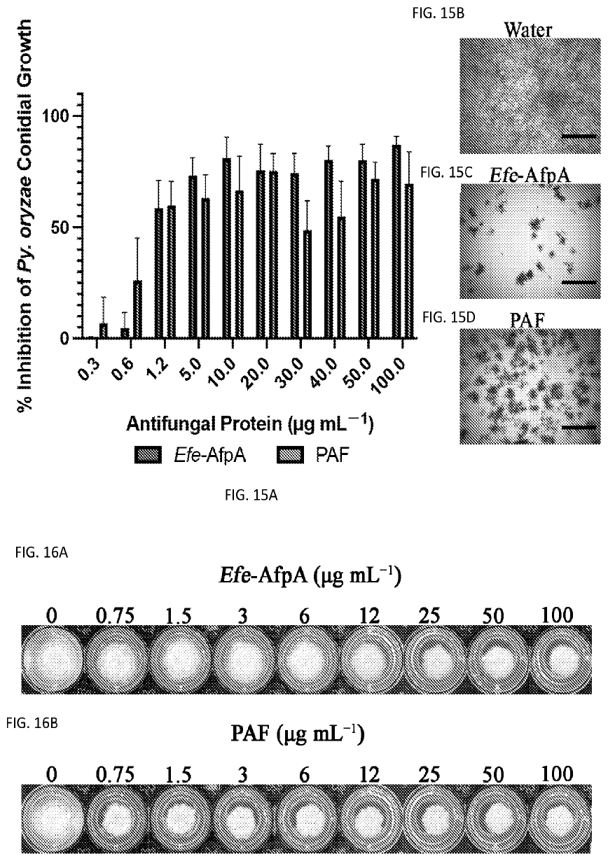

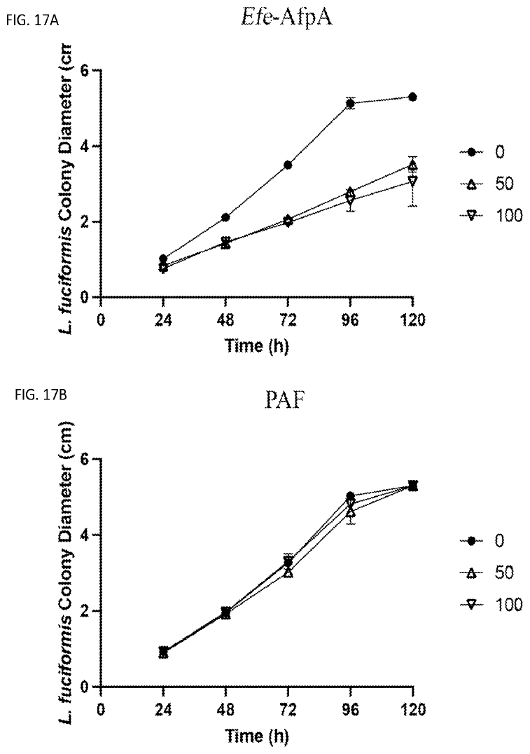

The well-studied antifungal protein PAF from Pe. chrysogenum has been tested against Py. oryzae. In previous studies, PAF treatment was ineffective, requiring a concentration greater than 200 g mL-1 (approximately 32 M) to completely inhibit growth [32,42]. Here, both Efe-AfpA and PAF did have activity against Py. Oryzae (FIG. 15, Table 1, FIG. 16).

Laetisaria fuciformis

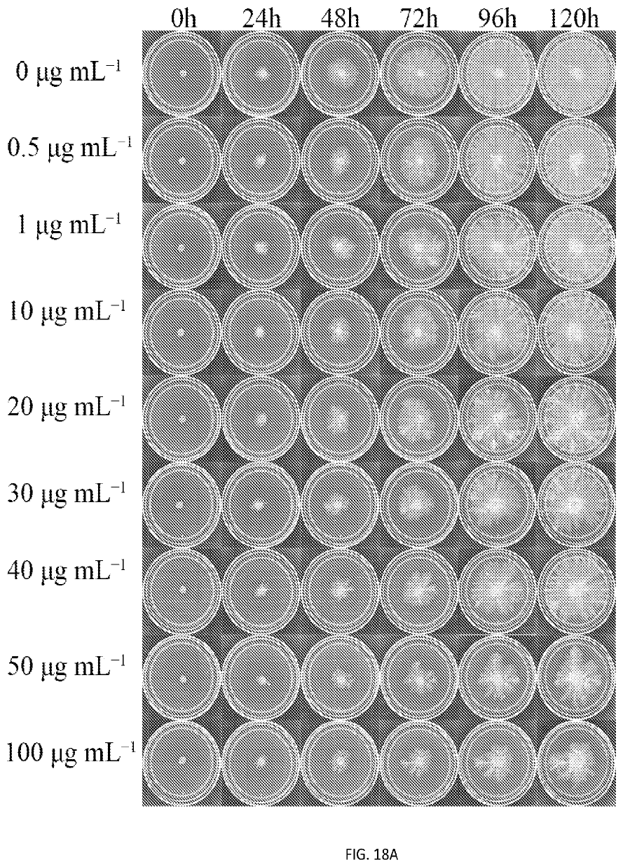

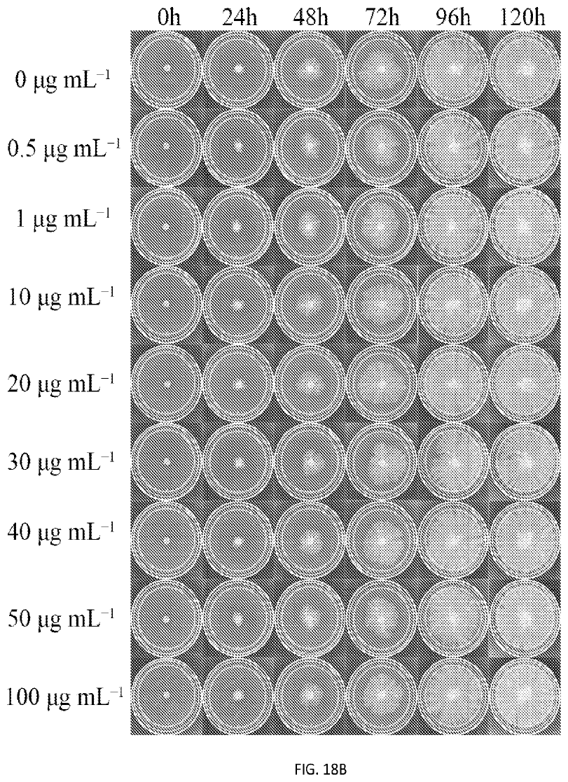

Laetisaria fuciformis causes red thread of turfgrasses, and is particularly damaging on Lolium perenne (perennial ryegrass) and Festuca rubra [33]. F. rubra has been shown to have enhanced tolerance to red thread when infected by the fungal endophyte E. festucae [19]. Efe-AfpA showed activity against L. fuciformis (FIGS. 17, Table 1, and FIGS. 18A-18B). PAF was ineffective against L. fuciformis.

### DISCUSSION

Here, we evaluated the antifungal activity of Efe-AfpA and PAF against additional important fungal plant pathogens in culture. Both proteins were effective at inhibiting growth of the pathogens, but there were some clear differences in their activity levels. Similarly, Efe-AfpA and PAF were previously found to differ in activity against Cl. jacksonii, the causal agent of dollar spot disease on turfgrasses [Example I]. Efe-AfpA inhibited the growth of Cl. jacksonii but PAF had no inhibitory activity against the pathogen. Antifungal proteins have been identified from numerous fungal species and differences in activity of other similar antifungal proteins have previously been reported [43,44]. Here, both Efe-AfpA and PAF had activity against all the Ascomycete fungi tested but the level of inhibition varied. Efe-AfpA had some activity against the Basidiomycete L. fuciformis whereas PAF had no activity.

The differences in activities of Efe-AfpA and PAF against the pathogens tested is likely due to the different environments in which E. festucae and Pe. chrysogenum exist and the roles of these antifungal proteins in the biology of two fungi. E. festucae is a fungal endophyte of grasses and does not exist in nature independently of its grass host. PAF was originally reported from Pe. chrysogenum [9], which is commonly found in indoor environments and is a food spoilage fungus [45]. It is unknown if the anti-fungal activities of the two proteins are their main functions. If so, their differences in activity could be the result of evolutionary changes resulting from different competing fungi present in their environments. However, additional functions for both Efe-AfpA and PAF have been suggested, which likely contribute to the sequence differences that result in activity differences. Efe-AfpA was suggested to be critical for the interaction of the fungus with its host grass [46]. PAF was proposed to have roles in conidiation and autophagy [47,48]. AfpB from Pe. digitatum is another similar antifungal protein, but is also active against its parent strain. AfpB may play a role in regulating the Pe. digitatum population, as well as other fungal populations [49].

New strategies are needed to combat plant pathogens and the development of antifungal proteins could provide some alternatives or complements to traditional fungicides. The data presented here indicate that specific antifungal proteins could be developed to target particular plant pathogens. Since Efe-AfpA had activity against numerous other important plant pathogens, in addition to its activity against Cl. jacksonii, it could be developed to target a broader range of destructive pathogens in both turfgrass and agronomic systems.

### ADDITIONAL REFERENCES

1. Zubrod, J. P.; Bundschuh, M.; Arts, G.; Bruhl, C. A.; Imfeld, G.; Knabel, A.; Payraudeau, S.; Rasmussen, J. J.; Rohr, J.; Scharmuller, A.; et al. Fungicides: An overlooked pesticide class?Environ. Sci. Technol. 2019, 53, 3347-3365.
2. Steinberg, G.; Gurr, S. J. Fungi, fungicide discovery and global food security. Fungal Genet. Biol. 2020, 144, 103476.
3. Morton V.; Staub, T. A. short history of fungicides. APSnet Features. doi: 10.1094/APSnetFeature-2008-0308 (accessed on 10-25-2022).
4. Nelson, R.; Wiesner-Hanks, T.; Wisser, R.; Ballint-Kurti, P. Navigating complexity to breed disease-resistant crops. Nat. Rev. Genet. 2018, 19, 21-33.
5. Sanchez-Martin, J.; Keller, B. Contribution of recent technological advances to future resistance breeding. Theor. Appl. Genet. 2019, 132, 713-732.
6. van Esse, H. P.; Reuber, T. L.; van der Does, D. Genetic modification to improve disease resistance in crops. New Phytol. 2020, 225, 70-86.
7. Green, K. K.; Stenberg, J. A.; Lankinen, A. Making sense of integrated pest management (IPM) in the light of evolution. Evol. Appl. 2020, 13, 1791-1805.
8. Ons, L.; Bylemans, D.; Thevissen, K.; Cammue, P. A. Combining biocontrol agents with chemical fungicides for inte-grated plant fungal disease control. Microorganisms 2020, 8, 1930.
9. Marx, F.; Haas, H.; Reindi, M.; Stoffler, G.; Lottspeich, F.; Redl, B. Cloning, structural organization and regulation of expression of the Penicillium chrysogenum paf gene encoding an abundantly secreted protein with antifungal activity. Gene 1995, 167, 167-171.
10. Huber, A.; Galgoczy, L.; Varadi, G.; Holzknecht, J.; Kakar, A.; Malanovic, N.; Leber, R.; Koch, J.; Keller, M. A.; Batta, G.; Toth, G. K.; Marx, F. Two small, cysteine-rich and cationic antifungal proteins from Penicillium chrysogenum: A comparative study of PAF and PAFB. Biochim. Biophys. Acta Biomembr. 2020, 1862, 183246.
11. Wnendt, S.; Ulbrich, N.; Stahl, U. Molecular cloning, sequence analysis and expression of the gene encoding an anti-fungal-protein from Aspergillus giganteus. Curr. Genet. 1994, 25, 519-523.
12. Kovacs, L.; Viragh, M.; Tako, M.; Papp, T.; Vagvolgyi, C; Galgoczy, L. Isolation and characterization of Neosartorya fischeri antifungal protein (NFAP). Peptides 2011, 32, 1724-1731.
13. Theis, T.: Wedde, M.; Meyer, V.; Stahl, U. The antifungal protein from Aspergillus giganteus causes membrane perme-abilization. Antimicrob. Agents Chemother. 2003, 47, 588-593.
14. Marx, F. Small, basic antifungal proteins secreted from filamentous ascomycetes: a comparative study regarding ex-pression, structure, function and potential application. App. Microbiol. Biotechnol. 2004, 65, 133-142.
15. Toth, L.; Boros, E.; Poor, P.; Ordog, A.; Kele, Z.; Varadi, G.; Holzknecht, J.; Bratschun-Khan, D.; Nagy, I.; Toth, G. K.; Rakhely, G.; Marx, F.; Galgoczy, L. The potential use of the Penicillium chrysogenum antifungal protein PAF, the designed variant PAFopt and its □-core peptide P□opt in plant protection. Microb. Biotechnol. 2020, 13, 1403-1414.
16. Leiter, E.; Gall, T.; Csernoch, L.; Pocsi, I. Biofungicide utilizations of antifungal proteins of filamentous ascomycetes: current and foreseeable future developments. BioControl 2017, 62, 125-138.
17. Braun, R. C.; Patton, A. J.; Watkins, E.; Koch, P. L.; Anderson, N. P.; Bonos, S. A.; Brilman, L. A. Fine fescues: A review of the species, their improvement, production, establishment, and management. Crop Sci. 2020, 60, 1142-1187.
18. Schardl, C. L.; Young, C. A.; Hesse, Ul; Amyotte, S. G.; Andreeva, K.; Calie, P. J.; Fleetwood, D. J.; Haws, D. C.; Moore, N.; Oeser, B.; et al. Plant-symbiotic fungi as chemical engineers: multi-genome analysis of the Clavicipitaceae reveals dynamics of alkaloid loci. PLoS Genet. 2013, 9(2): e1003323.
19. Bonos, S. A.; Wilson, M. M.; Meyer, W. A.; Funk, C. R. Suppression of red thread in fine fescues through endo-phyte-mediated resistance. Appl. Turfgrass Sci. 2005, 10, 1094.
20. Clarke, B. B.; White. J. F. Jr.; Hurley, R. H.; Torres, M. S.; Sun, S.; Huff, D. R. Endophyte-mediated suppression of dollar spot disease in fine fescues. Plant Dis. 2006, 90, 994-998.
21. Tian, Z.; Wang, R.; Ambrose, K. V.; Clarke, B. B.; Belanger, F. C. The Epichloë festucae antifungal protein has activity against the plant pathogen Sclerotinia homoeocarpa, the causal agent of dollar spot disease. Sci. Rep. 2017, 7:5643.
22. Heineck, G. C.; Qiu, Y.; Ehlke, N. J.; Watkins, E. The fungal endophyte Epichloë festucae var. lolii plays a limited role in mediating crown rust severity in perennial ryegrass. Crop Sci. 2020, 60, 1090-1104.
23. Ambrose, K. V.; Belanger, F. C. SOLiD-SAGE of endophyte-infected red fescue reveals numerous effects on host tran-scriptome and an abundance of highly expressed fungal secreted proteins. PLoS ONE 2012, 7, e53214.
24. Schardl, C. L.; Scott, B. Recommendations for gene nomenclature for Epichloe species and related Clavicipitaceae. In Epichloae, Endophytes of Cool Season Grasses: Implications, Utilization and Biology; Young, C. A., Aiken, G. E., McCulley, R. L., Strickland, J. R., Schardl, C. L., Eds.; The Samuel Roberts Noble Foundation, Ardmore, Oklahoma, USA, 2012; pp. 84-87.
25. Fardella, P. A.; Tian, Z.; Clarke, B. B.; Belanger, F. C. The Epichloë festucae antifungal protein Efe-AfpA protects creeping bentgrass (Agrostis stolonifera) from the plant pathogen Clarireedia jacksonii, the causal agent of dollar spot disease. J. Fungi 2022, 8.
26. Sonderegger, C.; Galgoczy, L.; Garrigues, S.; Fizil, A.; Borics, A.; Manzanares, P.; Hededus, N.; Huber, A.; Marcos, J. F.; Batta, G.; Marx, F. A Penicillium chrysogenum-based expression system for the production of small, cysteine-rich antifungal proteins for structural and functional analyses. Microb. Cell Factories 2016, 15, 192.
27. Williamson, B.; Tudzynski, B.; Tudzynski, P.; Van Kan, J. A. L. Botrytis cinerea: The cause of grey mould disease. Mol. Plant Pathol. 2007, 8, 561-580.
28. Dean, R.; Van Kan, J. A. L.; Pretorius, Z. A.; Hammond-Kosack, K. E.; Di Pietro, A.; Spanu, P. D.; Rudd, J. J.; Dickman, M.; Kahmann, R.; Ellis, J.; et al. The top 10 fungal pathogens in molecular plant pathology. Mol. Plant Pathol. 2012, 13, 414-430.
29. Weiberg, A.; Wang, M.; Lin, F. M.; Zhao, H.; Zhang, Z.; Kaloshian, T.; Huang, H. Da; Jin, H. Fungal small RNAs suppress plant immunity by hijacking host RNA interference pathways. Science 2013, 342, 118-123.
30. Kaiserer, L.; Oberparleiter, C.; Weiler-Görz, R.; Burgstaller, W.; Leiter, E.; Marx, F. Characterization of the Penicillium chrysogenum antifungal protein PAF. Arch. Microbiol. 2003, 180, 204-210.
31. Marx, F.; Binder, U.; Leiter, É.; Pócsi, I. The Penicillium chrysogenum antifungal protein PAF, a promising tool for the development of new antifungal therapies and fungal cell biology studies. Cell. Mol. Life Sci. 2008, 65, 445-454.
32. Garrigues, S.; Gandia, M.; Popa, C.; Borics, A.; Marx, F.; Coca, M.; Marcos, J. F.; Manzanares, P. Efficient production and characterization of the novel and highly active antifungal protein AfpB from Penicillium digitatum. Sci. Rep. 2017, 7.
33. Tredway, L. P.; Tomaso-Peterson, M.; Kerns, J. P.; Clarke, B. B. Compendium of Turfgrass Diseases (Fourth). 2022. APS Press, St. Paul, MN, USA.
34. Crouch, J. A.; Dawe, A.; Aerts, A.; Barry, K.; Churchill, A. C. L.; Grimwood, J.; Hillman, B. I.; Milgroom, M. G.; Pangilinan, J.; Smith, M.; et al. Genome sequence of the chestnut blight fungus Cryphonectria parasitica EP155: A fundamental resource for an archetypical invasive plant pathogen. Phytopathology 2020, 110, 1180-1188.
35. Newhouse, A.; Powell, W. Intentional introgression of a blight tolerance transgene to rescue the remnant population of American chestnut. Conserv. Sci. Pract. 2020, 3.
36. Yue, Q.; Miller, C. J.; White, J. P.; Richardson, M. D. Isolation and characterization of fungal inhibitors from Epichloë festucae. J. Agric. Food Chem. 2000, 48, 4687-4692.
37. King, R.; Urban, M.; Hammond-Kosack, M. C. U.; Hassani-Pak, K.; Hammond-Kosack, K. E. The completed genome sequence of the pathogenic ascomycete fungus Fusarium graminearum. BMC Genomics 2015, 16.
38. Leplat, J.; Friberg, H.; Abid, M.; Steinberg, C. Survival of Fusarium graminearum, the causal agent of Fusarium Head Blight. a review. Agron. Sustain. Dev. 2013, 33, 97-111.
39. Luo, J.; Zhang, N. Magnaporthiopsis, a new genus in Magnaporthaceae (Ascomycota). Mycologia 2013, 105, 1019-1029.
40. Skamnioti, P.; Gurr, S. J. Against the grain: Safeguarding rice from rice blast disease. Trends Biotechnol. 2009, 27, 141-150.
41. Vines, P. L.; Daddio, R. M.; Luo, J.; Wang, R.; Murphy, J. A.; Zhang, N.; Clarke, B. B.; Meyer, W. A.; Bonos, S. A. Pyricularia oryzae incites gray leaf spot disease on hard fescue (Festuca brevipila). Int. Turfgrass Soc. Res. J. 2022, 14, 997-1002.
42. Vila, L.; Lacadena, V.; Fontanet, P.; Martinez del Pozo, A.; San Segundo, B. A Protein from the mold Aspergillus gi-ganteus is a potent inhibitor of fungal plant pathogens. Mol. Plant-Microbe Interact. 2001, 14, 1327-1331.
43. Gandia, M.; Kakar, A.; Giner-Llorea, M.; Holzknecht, J.; Martinez-Culebras, P.; Galgoczy, L.; Marx, F.; Marcos, J. F.; Manzanares P. Potential of antifungal proteins (AFPs) to control Penicillium postharvest fruit decay. J. Fungi 2021, 7, 449.
44. Martinez-Culebras, P. V.; Gandia, M.; Boronat, A.; Marcos, J. F.; Manzanares, P. Differenial susceptibility of mycotox-in-producing fungi to distinct antifungal proteins (AFPs). Food Microbiol. 2021, 97, 103760.
45. Houbraken, J.; Frisvad, J. C.; Samson, R. A. Fleming's penicillin producing strain is not Penicillium chrysogenum but P. rubens. IMA Fungus 2011, 2, 87-95.
46. Wang, R.; Luo. S.; Clarke, B. B.; Belanger, F. C. The Epichloe festucae antifungal protein Efe-AfpA is also a possible effector protein required for the interaction of the fungus with its host grass Festuca rubra subsp. rubra. Microorganisms 2021, 9:140.
47. Hegedus, N.; Sigl, C.; Zadra, I.; Pocsi, I.; Marx, F. The paf gene product modulates asexual development in Penicillium chrysogenum. J. Basic Microbiol. 2011, 51, 253-262.
48. Kovacs, B., Hegedus, N., Balint, M., Szabo, Z., Emri, T., Kiss, G., Antal, M., Pocsi, I., Leiter, E. Penicillium antifungal protein (PAF) is involved in the apoptotic and autophagic processes of the producer Penicillium chrysogenum. Acta Microbiol, Immunol. Hung. 2014, 61:379-388.
49. Bugeda, A.; Garrigues, S.; Gandia, M.; Manzanares, P.; Marcos, J. F.; Coca, M. The antifungal protein AfpB induces regulated cell death in its parental fungus Penicillium digitatum. MSphere 2020, 5: e00595-20.

### Example III

### Expression of Antifungal Proteins in Plant, Fungal, or Bacterial Cells

The information herein above can be applied to generate transgenic plant, fungal and/or bacterial cells that produce the antifungal proteins described herein. In certain embodiments, the antifungal protein is Efe-AfpA. A preferred embodiment of the cells are transformed using particle bombardment, protoplast fusion, or agrobacterium transformation. The cells are transformed such that the antifungal protein coding sequence is downstream of the plant secretion sequence and the expression is driven by a strong plant promoter sequence. In certain embodiments, the promoter and targeting sequence are the sequences used in other Epichloe species. In certain embodiments, the introns are removed from the antifungal protein prior to transformation. In certain embodiments, the transformation methods described in Example I for the transformation of E. coli, P. pastoris, and/or P. chrysogenum can be used to transform plant, fungal and/or bacterial cells.

The transformants can then be analyzed using PCR to confirm the presence of the desired antifungal protein. PCR-positive cells can then be further analyzed by Northern blots for gene expression.

The antimicrobial cells can be extracted from the transformed cells and administered to plants of interest. In certain embodiments, the purification methods described in Example I for the purification of Efe-AfpA from E. coli, P. pastoris, and/or P. chrysogenum can be used to extract the protein from the transformed cells. In certain embodiments, the extracted antimicrobial protein inhibits the growth of a target organism infecting the plant of interest by at least 90%. In other embodiments, the extracted antimicrobial proteins inhibit the growth of the target organism by 100%. In certain embodiments, the extracted antifungal proteins prevent the target organism from growing on the plant. In certain embodiments, the target organism includes one or more of B. cinerea, Co. cereale, Cr. Parasitica, F. graminearum, P. oryzae, L. fuciformis. In certain embodiments, the antifungal protein is Efe-AfpA.

In certain embodiments, the transformed fungal and/or bacterial cells are applied directly to a plant of interest. In certain embodiments, the fungal and/or bacterial cells inhibit the growth of a target organism infecting the plant of interest by at least 40%. In certain embodiments, the fungal and/or bacterial cells inhibit the growth of a target organism infecting the plant of interest by at least 50%. In certain embodiments, the fungal and/or bacterial cells inhibit the growth of a target organism infecting the plant of interest by at least 60%. In certain embodiments, the fungal and/or bacterial cells inhibit the growth of a target organism infecting the plant of interest by at least 70%. In certain embodiments, the fungal and/or bacterial cells inhibit the growth of a target organism infecting the plant of interest by at least 80%. In certain embodiments, the fungal and/or bacterial cells inhibit the growth of a target organism infecting the plant of interest by at least 90%. In other embodiments, the plant cells inhibit the growth of the target organism by 100%. In certain embodiments, the antifungal proteins produced by the transformed cells prevent the target organism from growing on the plant. In certain embodiments, the target organism includes one or more of B. cinerea, Co. cereale, Cr. Parasitica, F. graminearum, P. oryzae, L. fuciformis. In certain embodiments, the antifungal protein is Efe-AfpA.

The transformed plant cells express the antifungal protein at a high enough level to inhibit the growth of a target organism. In certain embodiments, the plant cells inhibit the growth of the target organism by at least 40%. In certain embodiments, the plant cells inhibit the growth of the target organism by at least 50%. In certain embodiments, the plant cells inhibit the growth of the target organism by at least 60%. In certain embodiments, the plant cells inhibit the growth of the target organism by at least 70%. In certain embodiments, the plant cells inhibit the growth of the target organism by at least 80%. In certain embodiments, the plant cells inhibit the growth of the target organism by at least 90%. In other embodiments, the plant cells inhibit the growth of the target organism by 100%. In certain embodiments, the antifungal proteins produced by the transformed cells prevent the target organism from growing on the plant. In certain embodiments, the target organism includes one or more of B. cinerea, Co. cereale, Cr. Parasitica, F. graminearum, P. oryzae, L. fuciformis. In certain embodiments, the antifungal protein is Efe-AfpA.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for increasing yield of Efe-AfpA antifungal protein from transgenic P. chrysogenum^{Efe-AfpA} comprising;
a) culturing conidia from P. chrysogenum^{Efe-AfpA} in high nutrient media for a suitable time period to produce mycelia without inducing Ef-AfpA expression;
b) harvesting and culturing the mycelia of step a) in a minimal low nutrient media for a suitable time period to induce robust Efe-AfpA expression and secretion into said low nutrient media,
c) centrifuging and filtering said low nutrient media to remove conida and excess debris;
d) dialyzing said filtered media of step c) on an ion exchange column;
e) eluting said Efe-AfpA with increasing salt concentrations to form an eluant; and
f) filtering and desalting said eluant via passage through filter, thereby isolating said Efe-AfpA antifungal protein.

2. The method of claim 1, wherein the P. chrysogenum has been transformed with a nucleic acid construct encoding Efe-AfpA.

3. The method of claim 1, wherein
a) said high nutrient media comprises Aspergillus nidulans Complete Media comprising 0.2% Peptone, 0.1% Yeast Extract, 0.1% N—Z Amine, 2% Glucose, 2% Salt Solution A, 0.1% Trace Element Solution B, wherein Solution A comprises 0.1% FeSO₄×7H₂O, 0.9% ZnSO₄, 0.04% CuSO₄, 0.01% MnSO₄, 0.01% H₃BO₃, 0.01% Na₂MoO₄ and Solution B comprises 1.3% ZnSO₄, 0.07% CuSO₄, 0.1% MnSO₄, 0.006% Na₂B₄O₇, 0.23% FeSO₄×7H₂O and Salt Solution A is 2.6% KCl, 2.6% MgSO₄, 7.6% KH₂PO₄, 0.2% Chloroform (v/v); or
b) said low nutrient media is Penicillium chrysogenum Minimal Media comprising 0.3% NaNO₃, 0.05% MgSO⁴×7H₂O, 0.05% KCl, 0.005% FeSO₄×7H₂O, 2% Sucrose, 2.5% 1 M Potassium Phosphate Buffer pH 5.8, 0.1% Trace Elements Solution A, 2% agar) plates supplemented with 200 μg mL⁻¹ Nourseothricin and 0.6 μg mL⁻¹ Pyrithiamine.

4. (canceled)

5. An Efe-AfpA containing composition produced by the method of claim 1 in a carrier suitable for application to target plants.

6. The composition of claim 5, wherein said carrier is a
a) solid carrier selected from peat, wheat, bran, vermiculite, clay, chitin, talc, bentonite, diatomaceous earth, fuller's earth, and pasteurized soil;
b) mineral carrier selected from kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate; or
c) a liquid carrier selected from vegetable oil, soybean oil, cottonseed oil, glycerol, ethylene glycol, polyethylene glycol, propylene glycol, polypropylene glycol.

7-8. (canceled)

9. The composition of claim 5, further comprising one or more of
a) a surfactant selected from Tween 20, Prefer 28 (Cenex), Surf-N(US), Inhance (Brandt), P-28 (Wilfarm), Patrol (Helena); Sun-It II (AmCy), MSO (UAP), Scoil (Agsco), Hasten (Wilfarm), Mes-100 (Drexel), Silwet L77 (UAP), Silikin (Terra), Dyne-Amic (Helena), Kinetic (Helena), Sylgard 309 (Wilbur-Ellis) and Century (Precision), and
b) one or more water dispersible concentrates, wettable powders and surfactants suitable for spraying or dripping on a target plant.

10. (canceled)

11. The composition of claim 5, further comprising at least one fungicide and/or at least one nematode controlling agent.

12. (canceled)

13. A method for treating, and, or inhibiting, microbial infection and/or enhancing resistance to a microbial infection comprising administering a composition according to claim 5 to a target plant.

14. The method of claim 13, wherein said microbial infection is a fungal infection.

15. The method of claim 13, wherein the antifungal protein is from Epichloe festucae, E. gansuensis var. inebrians. E. baconii, E. aotearoae, and E. coenophiala.

16. (canceled)

17. The method of claim 13, wherein said method comprises administering the antifungal protein composition to the leaves of said plant or the soil.

18. (canceled)

19. The method of claim 13, wherein said method comprises administering a composition comprising the Epichloe endophyte to the plant.

20. The method of claim 13, wherein said fungal disease is dollar spot disease or red thread disease.

21. The method of claim 13, further comprising the administration of at least one fungicide and/or at least one nematode controlling agent.

22. (canceled)

23. A method for increasing yield of Efe-AfpA antifungal protein from transgenic Penicillium sp transduced with a nucleic acid encoding Efe-AfpA;
a) culturing conidia from said transduced Penicillium sp. in high nutrient media for a suitable time period to produce mycelia without inducing Efe-AfpA expression;
b) harvesting and culturing the mycelia of step a) in a minimal low nutrient media for a suitable time period to induce robust Efe-AfpA expression and secretion into said low nutrient media,
c) centrifuging and filtering said low nutrient media to remove conida and excess debris;
d) dialyzing said filtered media of step c) on an ion exchange column;
e) eluting said Efe-AfpA with increasing salt concentrations to form an eluant; and
f) filtering and desalting said eluant via passage through filter, thereby isolating said Efe-AfpA antifungal protein.

24. The method of claim 23, wherein an endogenous antifungal protein has been deleted or mutated in said Penicillium sp.

25. The method of claim 23, wherein the antifungal protein inhibits growth of one or more of B. cinerea, Co. cereale, Cr. parasitica, F. graminearum, Py. oryzae, and L. fuciformis.

26. A transgenic plant, transgenic fungus, or transgenic bacteria comprising an antifungal protein from Epichloe festucae, E. gansuensis var. inebrians. E. baconii, E. aotearoae, and E. coenophiala.

27.-28. (canceled)

29. The transgenic plant, the transgenic fungus, or the transgenic bacteria of claim 26, wherein the antifungal protein is Efe-AfpA from Epichloe festucae.

30. The transgenic plant, the transgenic fungus, or the transgenic bacteria of claim 26 wherein
a) the antifungal protein inhibits growth of one or more of B. cinerea, Co. cereale, Cr. parasitica, F. graminearum, Py. oryzae, and L. fuciformis;
b) the plant, fungus, or bacteria has been transformed with a nucleic acid construct encoding Efe-AfpA; or
c) the plant, fungus, or bacteria has been transformed using polyethylene glycol mediated protoplast transformation, lipofection, nucleofection, microinjection, biolistics, virosomes, liposomes, immunoliposomes, polycation or lipid:nucleic acid conjugates, naked DNA, artificial virions, or agent-enhanced uptake of DNA.

31.-32. (canceled)
